(19) United States
(12) Patent Application Publication
(10) Pub. No.: US 2011/0028859 A1
(43) Pub. Date: Feb. 3, 2011

(54) Methods, Systems and Devices for Monitoring a Target in a Neural System and Facilitating or Controlling a Cell Therapy

(75) Inventor: Martin Tsupon Chian, Los Altos, CA (US)

Correspondence Address: NEUROPACE, INC., 1375 SHOREBIRD WAY, MOUNTAIN VIEW, CA, 94043, US

(73) Assignee: NeuroPace, Inc., Mountain View, CA (US)

(21) Appl. No.: 12/552,247

(22) Filed: Sep. 1, 2009

Related U.S. Application Data
(60) Provisional application No. 61/230,677, filed on Jul. 31, 2009.

(51) Int. Cl. A61B 5/05

(52) U.S. Cl. 600/554

(57) ABSTRACT

Methods, systems and devices for monitoring a target in a neural system involve delivering inputs to and recording output responses from the neural system and obtaining I/O characterizations of the neural system both before and during or after a change is introduced to the system, for example, a change associated with a stem cell therapy. Baseline and therapeutic I/O characterizations are compared, and interpretations of the results of the comparison may be used to adjust some aspect of the therapy to facilitate it or control it. An implantable neurostimulator with the capability of delivering electrical stimulation to an electrode and of measuring and recording responses sensed from electrodes, where the electrodes are strategically positioned at or near the target, can be used to deliver an electrical stimulation input and record the output responses for the I/O characterizations. An implantable neurostimulator also may be used to affect a cell therapy by delivering electrical stimulation to the neural system to encourage stem cell differentiation.

### CROSS-REFERENCE TO RELATED APPLICATIONS

This application claims the benefit of priority to U.S. Provisional Patent Application Ser. No. 61/230,677, filed Jul. 31, 2009, which is incorporated herein by reference.

### BACKGROUND

1. Technical Field

This disclosure generally relates to methods, systems and devices for monitoring a target in a neural system before and during or after a change has been introduced to the neural system, for example, to assess the efficacy of a therapy associated with the change. More particularly, the monitoring includes comparing baseline and post-baseline (“therapeutic”) characterizations of the neural system to determine what impact, if any, the change has had on the neural system. This disclosure further relates to methods, systems and devices for facilitating or controlling a cell therapy based on a change that is introduced to a monitored neural system.

2. Background

Various forms of cell therapies are being researched for use in regenerative medicine (e.g., to treat a broad range of conditions including neurological disorders, cardiovascular and other diseases, such as diabetes). Cell therapies are also under consideration to improve the efficiency and efficacy of focused drug therapy to areas of the body, and to repair or strengthen areas of tissue such as brain tissue.

Stem cells are desired candidates for use in cell therapies because of their self-renewing properties and their capacity to differentiate into any cell type. There are multiple types of stem cells, including embroyonic stem cells (derived from blastocysts); fetal stem cells (taken from fetuses) and cord blood stem cells (isolated from umbilical cords); adult stem cells (found in tissues of adult humans); and stem cells derived from animals other than humans. Different types of stems cells have different capacities to differentiate, for example, embroyonic stem cells can differentiate into almost any other type of cell in the body (pluripotent), whereas adult stem cells including neural progenitor cells (NPCs) have less potency than embryonic stem cells, and thus only have the capacity to differentiate into one cell type (unipotent) or a fixed number of cell types (multipotent).

NPCs are essentially an intermediate stem cell type with the potential to differentiate, for example, in the nervous system, into a variety of cell types such as neurons and neuroglia or glial cells (i.e., non-neuronal cells that may provide any of support and nutrition, maintenance of homeostasis, myelin formation, and participation in signal transmission in the nervous system). (Astrocytes and oligodendrocytes are two subtypes of glial cells). NPCs also have the potential to renew themselves (i.e., by dividing into more NPCs.)

Cell therapies being studied for possible use in treating neurological disorders include those that rely on differentiation of stem cells into neuronal cells that are intended to repair or strengthen existing neural populations, neural circuits or portions of neural circuits. A therapy may involve implanting stem cells into the site or sites at which differentiation is desired or encouraging adult stem cells that already are present in a patient's body (endogenous stem cells) to differentiate or to migrate from one location to another and then differentiate.

A cell therapy may be used to supplement or improve the body's natural capacity to repair itself (research has shown that the development of new neurons from NPCs, in a process called neurogenesis, is disrupted when certain disorders, such as Parkinson's disease, epilepsy, and cancer, are present).

Being able to monitor a target of cell therapy, either directly or indirectly, would be desirable in order to, for example, assess whether the therapy is being or has been effective or whether it has resulted in undesirable or unintended consequences, such as formation of a teratoma (a type of tumor that is associated with stem cell differentiation or mutation). Current research techniques include using “markers” or unique molecules, typically made visible under various imaging, to track where stem cells have migrated or whether stem cells have differentiated into one cell type or another, but these techniques may not provide timely feedback as to whether a given cell therapy has been effective and/or requires adjustment in order to be effective and/or not results in unintended consequences.

Accordingly, it would be desirable to have practical and easily implemented devices, systems and methods for monitoring what is happening during or after a particular course of cell therapy in order to assess whether the therapy is effective or whether it is resulting in unintended consequences. For example, it would be desirable to have systems, devices and methods for monitoring a target in the system of a patient (e.g., a population of neurons, a known or suspected neural circuit or a portion thereof) to assess whether a cell therapy is having a desired effect. In particular, it would be desirable to characterize the target using computational neuroscience techniques and/or systems-theory based techniques to obtain a baseline, introduce a change in the form of a cell therapy that is intended to affect the target, characterize the target again during or after the cell therapy, and then compare the baseline characterization to the therapeutic characterization to determine whether the intended effect is occurring or has occurred.

It further would be desirable to have devices, systems and methods that can be used to control or facilitate the cell therapy itself, for example, to provide a form of stimulation to encourage stem cell differentiation or to modulate stimulation to promote or discourage a particular result.

The present disclosure describes methods, systems, and devices for performing one or more of these functions.

### SUMMARY

Before the present methods, systems and devices are described, it is to be understood that this disclosure is not limited to the particular methods, systems and devices described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “sensor” is a reference to one or more sensors and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention.

In an embodiment, a method for monitoring a target in a neural system includes choosing a target to monitor (e.g., based on a neurological examination, imaging, or other diagnostic testing); choosing a technique with which to acquire an input/output characterization of the neural system (e.g., linear or nonlinear, whether to use a systems model such as a feedback model, an addition model, or a cascade model, and whether to isolate one or more subsystems from other subsystems); choosing one or more inputs for the characterizations; applying the input(s) to the neural system and measuring the output and calculating the characterizations; and comparing one characterization to another (e.g., comparing a characterization undertaken before a change is introduced to the neural system (i.e., a baseline characterization) to a characterization undertaken during or after the change is introduced (i.e., a therapeutic characterization and/or comparing one therapeutic characterization to another therapeutic characterization of the same system).

In other embodiments, the method further includes facilitating a cell therapy by introducing a change to the system in the form of a cell therapy (e.g., implanting stem cells; applying a form of stimulation to implanted or endogenous stem cells to encourage their differentiation into a desired cell type and/or applying a form of stimulation to encourage strengthening of neural connections or inhibition of neural connections); and based on comparisons of various characterizations, determining whether to adjust a cell therapy to improve the results. For example, if the comparison results are above a predetermined threshold or outside of a predetermined range of values, then the method includes recommending an action or instigating an action to adjust the therapy in some fashion (e.g., stop the therapy or modify the parameters pursuant to which the therapy is delivered).

In still further embodiments, a system for monitoring a target in a neural system of a human patient before and after a change is introduced to the system includes an implantable sensor for sensing electrical activity (or for measuring another electrophysiological parameter) from a first location in the brain, an implantable device in communication with the sensor configured to measure a response of the sensor to a predetermined input, process the response and the input to calculate a plurality of input/output characterizations of the neural system based on each measured output response and the predetermined input and to communicate the characterizations to an external component, and a comparator to generate a comparison result from a baseline input/output characterization and a therapeutic input/output characterization or from two therapeutic characterizations acquired at different times.

In another embodiment, a system for monitoring a target in a neural system and facilitating a cell therapy includes an implantable sensor for sensing electrical activity (or for measuring another electrophysiological parameter) from a first location in the brain, an implantable device in communication with the sensor configured to measure a response of the sensor to a predetermined input, process the response and the input to calculate a plurality of input/output characterizations of the neural system based on each measured output response and the predetermined input and to communicate the characterizations to an external component, and a comparator to generate a comparison result from a baseline input/output characterization and a therapeutic input/output characterization or from two therapeutic characterizations acquired at different times, and components for adjusting a cell therapy based on the results of the comparison.

In other embodiments, a device for monitoring a target in a neural system includes component(s) for recording and temporarily storing information corresponding to an electrophysiological signal measured from a patient's brain; component(s) for accomplishing signal processing on the measured signal; component(s) for providing a stimulation input to modulate the signal measured from the patient's brain; and component(s) for communicating with external devices to accomplish one or more I/O characterizations based on the measured signal and the stimulation input and to compare the results of one I/O characterization to one or more other characterizations.

In still further embodiments, a device for monitoring a target in a neural system and for facilitating a cell therapy includes component(s) for recording and temporarily storing information corresponding to an electrophysiological signal measured from a patient's brain; component(s) for accomplishing signal processing on the measured signal; component(s) for providing a stimulation input to modulate the signal measured from the patient's brain; component(s) for communicating with external devices to accomplish one or more I/O characterizations based on the measured signal and the stimulation input and to compare the results of one I/O characterization to one or more other characterizations; and component(s) for adjusting a cell therapy being monitored with the I/O characterizations.

### BRIEF DESCRIPTION OF THE DRAWINGS

Aspects, features, benefits and advantages of the embodiments described herein will be apparent with regard to the following description, appended claims and accompanying drawings where:

FIG. 1 is a flow diagram of a method according to an embodiment for monitoring a target and, optionally, for adjusting a cell therapy based on the results of the therapy;

FIG. 2A is a graphical representation of a constant frequency pulse train input that may be used in characterizing neural systems.

FIG. 2B is a graphical representation of a paired pulse train input that may be used in characterizing neural systems.

FIG. 2C is a graphical representation of a random pulse train input that may be used in characterizing neural systems

FIG. 2D is a graphical representation of a biphasic pulse train input that may be used in characterizing neural systems.

FIGS. 3A and 3B are representations of a time-invariant linear system.

FIG. 4A is a graphical illustration of a paired pulse input used in obtaining a baseline characterization of a linear system.

FIG. 4B is a graphical illustration of a linear system output response based on the paired pulse input of FIG. 4A.

FIG. 4C is a graphical illustration of an impulse response based on the input of FIG. 4A and the output of FIG. 4B.

FIG. 4D is a graphical illustration of a transfer function based on the input of FIG. 4A and the output of FIG. 4B.

FIG. 5A is a graphical illustration of a paired pulse input used in obtaining a therapeutic characterization of the linear system of FIGS. 4A-4D, after a change has been introduced to the system.

FIG. 5B is a graphical illustration of a linear system output response based on the paired pulse input of FIG. 5A.

FIG. 5C is a graphical illustration of an impulse response based on the input of FIG. 5A and the output of FIG. 5B.

FIG. 5D is a graphical illustration of a transfer function based on the input of FIG. 5A and the output of FIG. 5B.

FIG. 6 is a representation of a nonlinear system.

FIG. 7A is a graphical illustration of a random pulse input used in obtaining a baseline characterization of a nonlinear system.

FIG. 7B is a graphical illustration of a nonlinear system output response based on the random pulse input of FIG. 7A.

FIG. 7C is a graphical illustration of a first order kernel in the time domain based on the input of FIG. 7A and the output of FIG. 7B.

FIG. 7D is a graphical illustration of the first order kernel of FIG. 7C in the frequency domain.

FIG. 7E is a graphical illustration of a second order kernel in the time domain based on the input of FIG. 7A and the output of FIG. 7B.

FIG. 7F is a graphical illustration of the second order kernel of FIG. 7E in the frequency domain.

FIG. 8A is a graphical illustration of a random pulse input used in obtaining a therapeutic characterization of the nonlinear system of FIGS. 7A-7F, after a change has been introduced to the system.

FIG. 8B is a graphical illustration of a nonlinear system output response based on the random pulse input of FIG. 8A.

FIG. 8C is a graphical illustration of a first order kernel in the time domain based on the input of FIG. 8A and the output of FIG. 8B.

FIG. 8D is a graphical illustration of the first order kernel of FIG. 8C in the frequency domain.

FIG. 8E is a graphical illustration of a second order kernel in the time domain based on the input of FIG. 8A and the output of FIG. 8B.

FIG. 8F is a graphical illustration of the second order kernel of FIG. 8E in the frequency domain.

FIG. 9A is a schematic illustration of a feedback model of a neural system.

FIG. 9B is a schematic illustration of an addition model of a neural system.

FIG. 9C is a schematic illustration of a cascade model of a neural system.

FIG. 10A is a schematic illustration of a feedback model of a neural system in which one subsystem “A” has been isolated from the rest of the system.

FIG. 10B is a schematic illustration of an addition model of a neural system in which one subsystem “A” has been isolated from the rest of the system.

FIG. 10C is a schematic illustration of a cascade model of a neural system in which one subsystem “A” has been isolated from the rest of the system.

FIG. 11 is a schematic illustration of a system for monitoring and/or delivering a cell therapy.

FIG. 12 is a flow diagram of a system for monitoring a cell therapy.

FIG. 13 is a flow diagram of a system for monitoring and adjusting a cell therapy.

FIG. 14 is a schematic illustration of a hippocampal structure in a human patient.

FIG. 15 is a schematic illustration of a systems model of a neural system corresponding to one or more neural pathways in the hippocampal structure of FIG. 14.

FIG. 16A is a schematic illustration of the systems model of FIG. 15 with one subsystem “I” of FIG. 15 eliminated. Overall system “C” can be characterized from input and output data.

FIG. 16B is a schematic illustration of the systems model of FIG. 16A showing isolation of subsystem “A” from subsystem “B”. Subsystem “A” can be characterized from input and output data.

FIG. 16C is a schematic illustration of the systems model of FIGS. 16A and 16B, showing isolation of a subsystem “B” from an overall system “C” and a subsystem “A.”. Subsystem “B” can be computed from mathematical derivations using characterizations of subsystem “A” and the overall system “C.”

FIG. 17A is a schematic illustration of a system for monitoring and facilitating a cell therapy in a hippocampal pathway.

FIG. 17B is a block diagram of a system for monitoring a neural target.

FIG. 17C is a block diagram of another system for monitoring a neural target.

FIG. 18A is schematic illustration of a systems model of a region of the hippocampus comprising a neural system including a target to be monitored.

FIG. 18B is a schematic illustration of the systems model of FIG. 18B showing a subsystem “A” isolated from a subsystem “B”.

FIG. 19 is a graphical representation of a form of electrical stimulus used in connection with a cell therapy to encourage differentiation of stem cells.

### DETAILED DESCRIPTION

Methods, systems and devices are described for monitoring a target in the central nervous system (CNS) whereby a baseline characterization of the target is compared to one or more therapeutic characterizations of the target to assess whether a form of cell therapy has had an effect and/or whether such effect is desirable. Therapeutic characterizations of the target that are undertaken at different times (e.g., during, just after and sometime after delivery of a cell therapy) also may be compared to monitor and/or assess the therapy. The characterizations may be enhanced by neurophsyiological modeling of the target. The form of therapy may be promoted, enhanced or otherwise facilitated by delivering a form of stimulation to modulate the behavior of stem cells and/or the target.

As used herein, “target” generally refers to the object of the monitoring that can be undertaken by the methods, systems and devices described herein. The target may comprise one or more of an individual neuron, a group of physically co-located or interconnected neurons; a group of nervous system cells of a particular cell type (e.g., neurons or cells) or functional classification (e.g., inhibitory, excitatory, or modulatory neurons); a brain circuit or part of a neural circuit that traverses a relatively large physical area of the brain (e.g., a hippocampal pathway); and/or any other logical grouping of cells or cell types in the brain or central nervous system. The target may be the same as or different from the object of a particular cell therapy. For example, stem cells may be implanted into a first site with the intent that the stem cells become established and differentiate into a desired cell type or types at that first site. Alternatively, and because of various factors, including the nature of the neuroanatomy, the brain circuitry, or any model(s) selected to use in monitoring the stem cell progress after implant, the target may be a second site that is more conducive to setting up monitoring than is the first site but which nevertheless permits inferences to be made as to whether a cell therapy has had an effect or a desired effect because of some relationship between the second site and the first site.

It is noted incidentally that the term “target” also may be used in a different context, more particularly, when referring to the neuron(s) affected by the action of another neuron, especially when neurons are classified in terms of their function. For example, an “excitatory neuron” is one that is often described as affecting its target neurons by activating receptors on the target cells that cause an increase in activity or firing rate; an “inhibitory neuron” is a neuron that affects its target neurons by interacting with receptors on the targets that cause a decrease in activity or firing rate; and a “modulatory neuron” is one that affects its target neurons by causing long-lasting effects not related to firing rate. Both uses of “target” may be found herein, and the particular meaning intended in any given case is expected to be clear from the context.

As used herein, a “neural system” refers to any physical or functional group of neurons the behavior of which may be predicted or approximated by empirically-derived results or with mathematics, for example, using linear or non-linear systems theory. “Neural system” may or may not also refer to something that is capable of representation by a model based on systems theory, for example, a feedback model, a cascade model, an addition model, etc., and which may or may not include or otherwise be definable by one or more subsystems. A “neural system” may also constitute a target or may be related to a target.

As used herein, a “characterization” or an “I/O characterization” refers to a mathematical relationship that is derived based on the output of a system in response to a predetermined the input to the system, wherein the derived mathematical relationship can be used to predict system behavior, i.e., for a given input, predict what will be the output. The mathematical relationship derived may be linear or non-linear in nature.

As used herein, a “baseline characterization” is a characterization that is derived before some change to the system is introduced, and a “therapeutic characterization” is a characterization that is derived either while the change is being introduced or at some time after the change is introduced (e.g., immediately after, an hour after, six months after, etc.)

The basic functional unit of the central nervous system is the neuron, which communicates information by sending impulses (chemical or electrical) across junctions or synapses between neurons. A typical neuron is a so-called “Type I” neuron and has a cell body or soma which contains the cell nucleus, a dendritic tree disposed around the cell body, a long thin axon covered by a myelin sheath that extends from the cell body and ends in branching terminals called axon terminals. Most of the input to the neuron is via the dendrites. The axons generally carry nerve signals away from the soma to the synapses of the axon terminals, where neurotransmitter chemicals are released in order to communicate with the cell's target neurons. (“Type II” neurons do not have axons.)

Neurons may be further classified in several ways: (1) according to their structure (e.g., unipolar, bipolar, multipolar (Golgi I and Golgi II)); (2) according to their function (e.g., excitatory neurons which evoke excitation of their target (object) neurons, inhibitory neurons which evoke inhibition of their target (object) neurons, and modulatory neurons which evoke effects more complex than excitation or inhibition): (3) according to their connectivity (e.g., afferent (conduct information from tissues and organs towards the CNS), efferent (conduct information from the CNS, also known as motor neurons), or interneurons (connect neurons to neurons within the CNS); (4) according to their discharge patterns (e.g., tonic (always active), phasic or bursting (fire in bursts), fast spiking (fast firing rates), and thin spiking (narrower action potentials as compared with other neurons); and (5) by the neurotransmitter they release (e.g., cholinergic, GABA-ergic, glutamatergic, and dopaminergic neurons). A target may be identified as a population of one of these types of neurons or, potentially, as a single example of a single cell within one of these neural cell types.

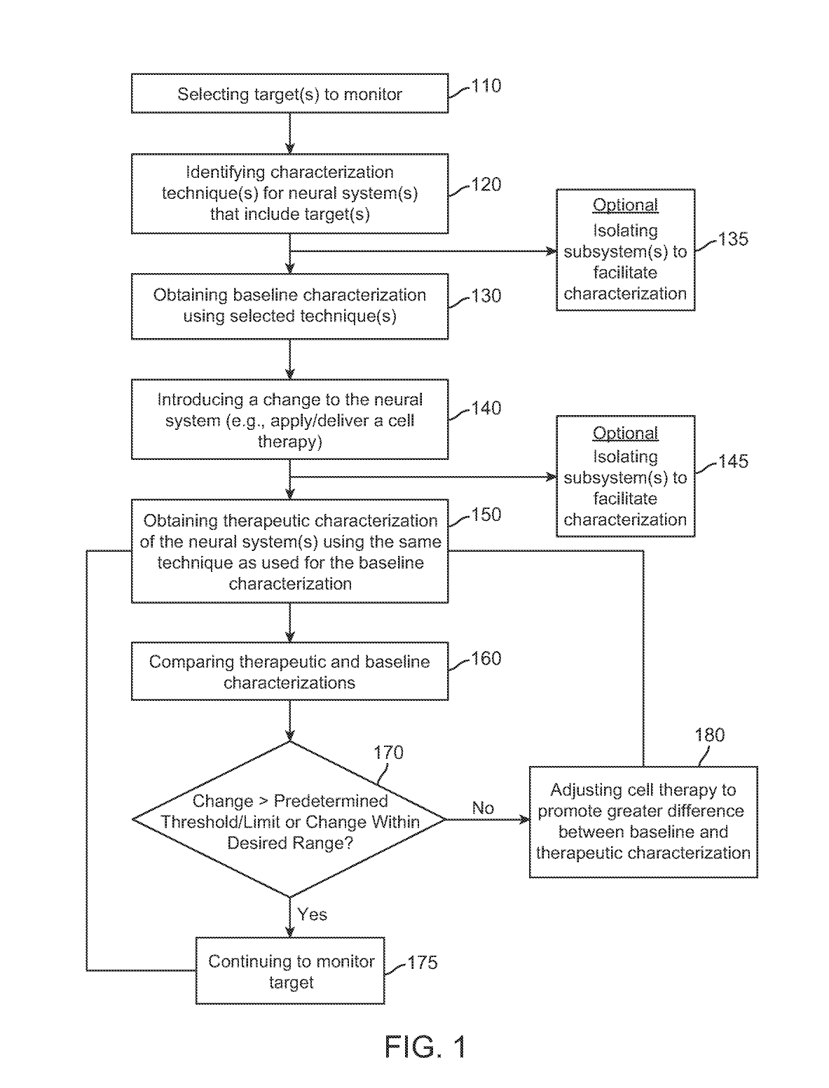

Referring now to FIG. 1, target selection 110 involves selecting one or more targets to monitor. Target selection may involve, among other things: (1) selecting a target that comprises a structure in the brain to which a cell therapy is to be delivered; (2) selecting a target that comprises a circuit or part of a circuit or neural pathway which a cell therapy is intended to repair or otherwise improve functionally or structurally; and (3) selecting a target that comprises a structure in the brain that is functionally or physically related to a structure in the brain to which a cell therapy is to be delivered.

Identifying the target in any of the categories identified above may be accomplished, in whole or in part, through a battery of diagnostic tests, such as clinically-administered neurological testing, imaging techniques and other techniques using feedback or evoked potentials using microelectrodes or macroelectrodes.

For example, a patient's symptoms initially may be explored with some basic neurophysiological tests to identify a suspected location of a population of neurons or a circuit that is damaged or functionally impaired by reason of a disease or other condition. Clinical tests can range from the simple to the complex. For example, in a clinical setting, a patient can be asked to perform a task that is known or suspected to be associated with normal or abnormal brain function in a certain region of the brain and, if the patient cannot perform the task and cannot fully or optimally perform the task (e.g., moving a limb through a range of normal range of motion, reciting a list of words from memory, etc.), then damage to the region might be assumed. Alternatively or additionally, the patient may be connected to external equipment (for example, with scalp electrodes or in-brain electrodes that are implanted acutely) that is capable of monitoring or recording signals associated with particular regions of the brain while the patient participates in a battery of clinical testing.

There also are multiple options for diagnostic imaging available, again ranging from the simple and traditional to the sophisticated and cutting-edge. For example, X-rays, MRI scans, or CT scans can be used to identify neural systems and/or pinpoint damage to those systems (e.g., to diagnose brain and spinal cord injuries by revealing areas of fracture, hemorrhage or certain kinds of tissue injury). Other imaging techniques, such as SPECT (single-photon emission computed tomography) or PET (positron emission tomography) can be used to detect changes due to brain injury by measuring brain cell metabolism as opposed to, for example, tissue density. Connectomics, such as the Brainbow technique and ATLUM (automatic tape-collecting lathe ultra microtome), also can be used to identify specific locations in or specific circuitry of the central nervous system.

In addition, clinical testing and imaging can be used in combination for diagnosis and identification and location of targets. For example, a patient may be asked to perform tasks or to try to recall information or think about certain things while undergoing a form of imaging, such as fMRI (functional magnetic resonance imaging).

In still other diagnostic approaches, microelectrode recording (MER) can be a precise method of localizing sites in the brain and can be used not only to locate a structure for diagnostic testing but also to place sensors for use, for example, in monitoring a target as described herein or devices for delivering a form of treatment, for example, for placing an electrode for delivering neuromodulation treatment to encourage stem cells to differentiate as described herein. For instance, microelectrode recording can be used to locate a deep brain structure, such as by passing the microelectrode through the basal ganglia and the thalamus until it reaches a predetermined final destination such as the substantia nigra (STN), globus pallidus (Gpi), or ventral intermediate nucleus (VIM).

Another technique for identifying locations in the nervous system is by using somatosensory evoked potentials (SSEPs) in which a somatosensory pathway is stimulated through strategically placed stimulation and recording electrodes, and the evoked potentials measured to locate areas that are functioning abnormally. Like the MER technique, the SSEP technique also is useful in guiding placement of sensors (e.g., electrodes) to sites in the brain for use in monitoring a target and/or for delivering stimulation as a form of cell therapy as described herein. Other techniques for identifying a target to be monitored will be apparent to those with skill in the art.

As noted above, a target may be identified as the site of damage itself, part of a circuit that is functioning abnormally by reason of the damage or other undesirable condition), or a physical structure or circuit that is somehow related to a damaged structure or circuit, such that by monitoring that physical structure or circuit, inferences or, in some cases, deductions may be made about the condition of the damaged structure or circuit.

A target may be a structure in an experimentally determined brain circuit or a portion or segment of such a circuit. For example, the limbic system is believed to regulate, among other things, emotions, as well as effect long-term memory and olfaction and is comprised of structures such as the amygdala, the hippocampus, the parahippocampal gyms, the cingulate gyrus, the formix, the hypothalamus, and the thalamus). The hippocampal pathways, portions of which are discussed in connection with an example below, are well-studied brain circuits that have been described in terms of such segments and inputs and outputs as the perforant path, the mossy fiber pathway and the collateral/associational commisural pathway, the granule cells, the dentate gyrus, the pyramidal cells and the perforant pathway. A brain circuit or network also may be defined in terms of a particular neurological disorder, such as a Huntington Disease Related Disorder circuit.

An example of a case in which the target selected to monitor is different than the site or portion of a circuit that is damaged or functioning abnormally might be as follows. A population of interneurons which have an inhibitory function (that is, are neurons that prevent their target neurons from firing) has been diagnosed as having been damaged or as functioning less than optimally. This particular interneuron population is spatially diverse in the region of the brain in which it is found (e.g., the hippocampus) and the total quantity of interneurons in the population is not large. Thus, it would be challenging to identify a location or locations in the brain at which to place sensors that could be relied upon to accurately record the response of the interneuron population to a form of cell therapy. However, the damage to the interneuron population is understood to allow another, associated population of neurons to fire more than those neurons ordinarily would fire if the interneuron population were functioning optimally. This associated population is in a location in the brain that is reasonably easy to record from, and the density of neurons in this population is sufficient to provide some level of confidence that the recorded signals will accurately reflect whether the firing rate of the neurons in this population has decreased, stayed the same, or increased. In this circumstance, the population of interneurons may be the population to which a form of cell therapy is delivered (e.g., the population in which stem cells are implanted with the hope that the stem cells will differentiate into inhibitory neurons); but the target selected to monitor to assess whether the form of cell therapy was effective might be the associated population, since it is a more practical target to monitor.

Another example of an indirect relationship between a target selected to monitor and a site at which treatment is delivered is where the target corresponds to neurons in a node of a neural pathway from which a measurable response might be expected when a cell therapy is delivered to a treatment site that affects the node but that is in another part of the same circuit or in another circuit.

Referring again to FIG. 1, a technique for acquiring characterizations of the target must be identified, so that monitoring can be accomplished by comparing a baseline characterization to a later obtained therapeutic characterization (or by comparing multiple therapeutic characterizations of the same target to each other). Identifying a characterization technique 120 may be informed by the nature of the target. For example, when mathematical or systems theory can be used to model the target structure, circuit or portion of a circuit, then the technique for monitoring desirably will take advantage of or otherwise be consistent with the theory in terms of the inputs and mathematics used for acquiring baseline and therapeutic characterizations.

In other cases, selection of a particular characterization technique may circumscribe a range of possible targets to monitor with the techniques. In still other cases, selection of both the characterization technique and the target to monitor with the characterizations may be driven by the limitations of the physical anatomy, for example, the possible locations at which electrodes for delivering inputs and recording outputs to accomplish the characterizations can be placed.

Some examples of characterization techniques are described below. Further, each of the selection of the characterization technique, the target to monitor and the introduction of inputs and recording of outputs for the characterization may be dictated, at least in part, by the desired manner of presentation of the results of the comparison of the baseline and therapeutic characterizations (or therapeutic-to-therapeutic characterizations, as the case may be).

In still other circumstances, the nature of the cell therapy will be a factor in selecting a characterization technique. For example, if the cell therapy will involve delivering electrical stimulation through an electrode to a site in the brain at which stem cells have been implanted, then it may be practical to choose a characterization technique that involves using an electrode to deliver electrical stimulation as an input with which to characterize a system including the target. With the foregoing in mind, some possible characterization techniques are described below.

It is widely known that temporal patterns determine the processing of information in the nervous system, so systems theory can beneficially be used to characterize neural systems. The input for a given input/output characterization of a neural system may be selected from a variety of different types of stimuli, such as electrical stimulation delivered through an electrode or electrode(s) implanted in or on the brain, stimuli derived from a neurological examination of a patient in a clinical setting, pharmacological stimulation directed to the target through a needle or a catheter, and other forms of stimulation such as optical, thermal, ultrasonic, auditory or visual.

The type of input(s) used in obtaining a characterization can be important to the usefulness of the measured output in obtaining a characterization. For example, the closer the input is to a naturally-occurring input to the neural system, the more likely it is that the characterization will reveal biologically meaningful insights. However, it may not always be practical to use an input that is close to a naturally-occurring because, for example, the required computations are too complex in view of component limitations or power consumption limitations.

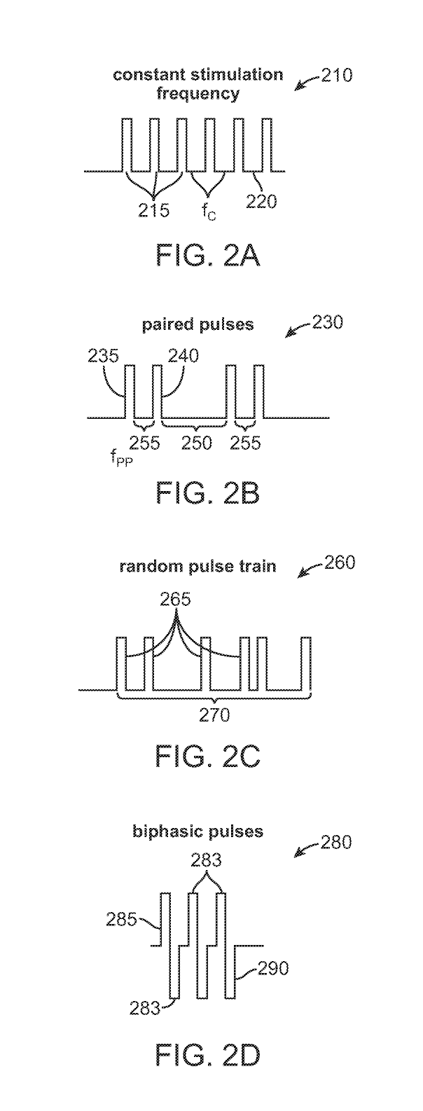

Referring now to FIGS. 2A-2D, examples of possible electrical stimuli for inputs to a neural system are illustrated.

FIG. 2A shows an input pulse train 210 where the pulses 215 are delivered with a constant frequency, f_{c} 220. FIG. 2B shows a paired pulse input 230, wherein pulses 235 and 240 have an interburst interval 250 and are delivered at a frequency, fₚₚ 255. It is a relatively simple matter to configure a pulse generator to deliver either a constant frequency pulse train 210 or a paired pulse input train 230. The output of a neural system based on a paired pulse input train 230 also provides an output that is relatively easy to interpret, because it can be assumed that the interactions reflected in the output response are caused by the specific pulses in a pair. Paired pulse inputs have been commonly used in neurophysiological research.

FIG. 2C shows a random pulse train 260, where pulses 265 are delivered randomly at a varying frequency, fᵣ 270. A random pulse train input provides a complex output, and the random pulses 265 are believed to better approximate naturally-occurring neural activity in the brain than do pulses delivered at a constant frequency or paired pulses. A random pulse train 260 thus may lead to more biologically meaningful insights into system behavior. Random pulse trains 260 also have been used in neurophysiological research, but interpreting the output that results from a random pulse train input can be computationally intensive and more challenging to render meaningful.

FIG. 2D shows an input 280 comprising biphasic pulses 283, each pulse 283 having a positive phase 285 and a negative phase 290. Biphasic pulse trains 280 are a form of a “charge-balanced” pulse train which are preferred over other types of pulses because they are less likely to damage the tissue at the electrode-to-tissue interface when applied chronically. For example, biphasic pulse trains have been used in the responsive neurostimulation therapy that is implemented in various implantable medical devices by NeuroPace, Inc. and is described in, inter alia, U.S. Pat. No. 6,016,449 to Fischell et al. for “System for the Treatment of Neurological Disorders” issued Jan. 18, 2000 and U.S. Pat. No. 6,810,285 to Pless et al. for “Seizure Sensing and Detection Using an Implantable Device” issued Oct. 26, 2004, both of which patents are hereby incorporated by reference as though set forth in full herein). Charge-balanced pulses can have different positive and negative phases, as long as the total positive and negative charges are the same. A charge-balanced pulse train input can be delivered at a constant frequency, in pairs with an interburst interval or randomly with a varying frequency as described for the non-biphasic pulses described with respect to FIGS. 2A-C above.

As will be apparent to those with skill in the art, electrical stimulation can excite neuronal synapses (excitatory response) or depress synapses (inhibitory response), depending on parameters such as the time between stimuli (i.e. the offset of one stimulus to the onset of another stimulus or “interstimulus interval”), and the amplitude and/or frequency of the pulses. For example, synaptic excitation or depression can occur when there are interactions between pulses in the target. Thus, it may be appropriate in certain cases to vary one or more pulse parameters in an electrical stimulation input (e.g., amplitude, interstimulus interval, etc.) to acquire a more meaningful characterization of a target.

Alternatively, the input for a characterization may be partially or entirely clinical (e.g., requiring the patient to perform a series of tasks (memorization and recall, moving limbs through a range of motion, auditory sounds or visual stimuli etc.). For example, in Parkinson's disease, a common test of bradykinesia (slowness of movement) is to measure how quickly a patient can tap their finger and thumb together. This test could be used as the input to establish a baseline characterization (e.g., recording an electrophysiological response to the test from a sensor and then calculating an I/O characterization based on the test and the recorded response) and repeated later for obtaining a therapeutic characterization. Clinical tests used to assess tremor, rigidity and postural instability could be used as well. Similarly, where the disorder being diagnosed and/or treated stroke, one could use common tests for stroke assessments as inputs, such as the ability to smile, move a limb, and speak. For example, a recording electrode could be positioned to record an electrophysiological response when a patient is instructed to try to move a limb. This recorded response could be identified as an input to a neural system. Another recording electrode may be positioned at a different location corresponding to an output of the neural system. The two recorded responses could be used to calculate an I/O characterization for the system. In still other cases, the input may be comprised in whole or in part of stimuli in a variety of other forms, such as a drug, sound, light or a thermal change.

Furthermore, the clinical input could be a raw auditory signal such as sound for characterizing the auditory system or light stimuli to characterize the visual system. These clinical inputs could be captured using internal or external equipment and used in characterization of a system or subsystems.

Referring now to FIG. 1 and FIGS. 3-8, identifying characterization techniques 120 and obtaining baseline characterizations 130 and therapeutic characterizations 150 based on both linear and nonlinear systems theory will be described.

According to systems theory, a discrete linear system is present when an output y[n] can be described with regards to its input x[n] and an impulse response function h[n] according to the convolution equation:

$$y [ n ] = {\sum }_{0}^{M - 1} h [ m ] x [ n - m ]$$

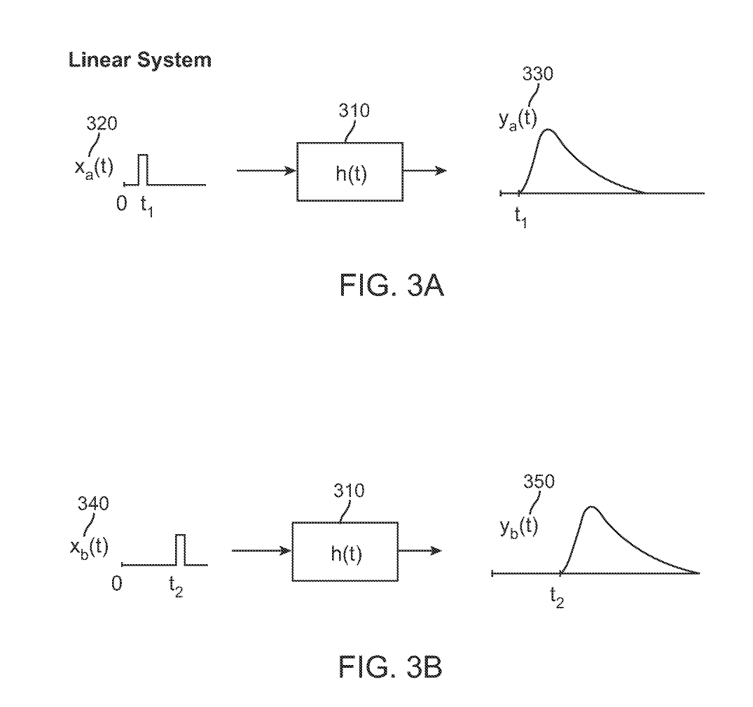

As illustrated in FIG. 3A, an impulse response h(t) 310 describes the transformation between an input xₐ(t) 320 to an output yₐ(t) 330. Further, and with reference to FIG. 3B, for a linear time-invariant system, the same input at a later time t₂, x_{b}(t) 340 (x_{b}(t)=xₐ(t+Δ)) generates the same output at that corresponding later time t₂, y_{b}(t) 350 (y_{b}(t)=yₐ(t+Δ)).

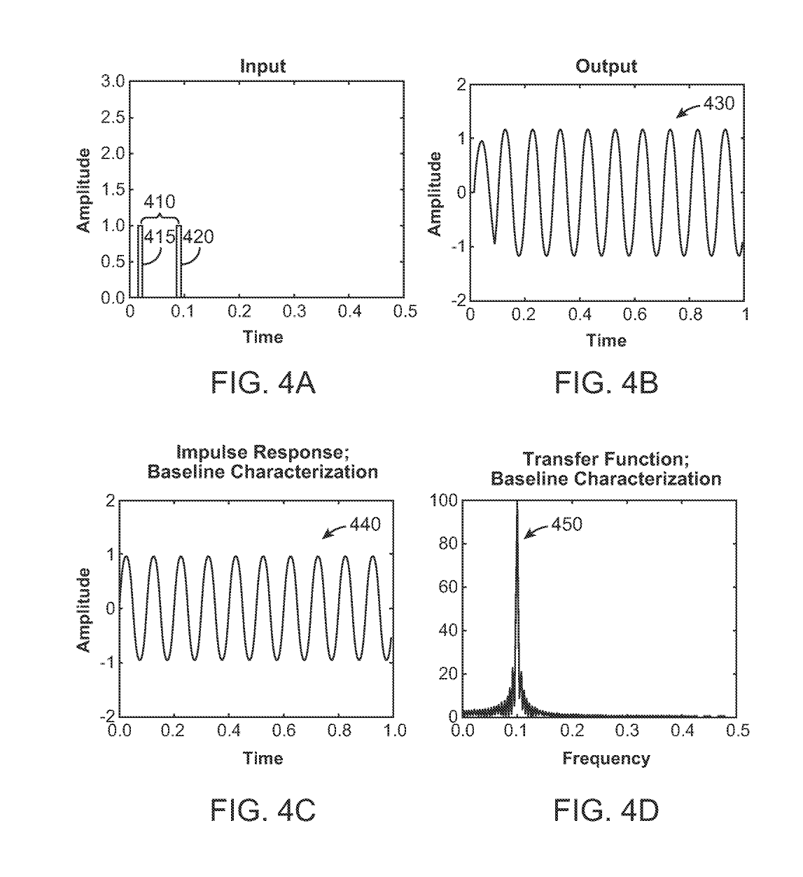

FIGS. 4A-4D graphically illustrate a possible baseline characterization for a neural system modeled as a simple time-invariant linear system (i.e., no subsystems). FIG. 4A illustrates a paired pulse input 410 comprising individual pulses 415, 420, which are graphically represented with respect to amplitude (y-axis) and time (x-axis).

FIG. 4B shows the output 430 of the linear system based on the paired pulse input 410. From the input and output of a linear system, an impulse response can be computed using the convolution theorem that states that convolution in the time domain is equivalent to multiplication in the frequency domain or:

Y(ω)=H(ω)X(ω)

Conversion back to the time domain from the frequency domain results in the impulse response. Thus, an impulse response is the mathematical function that describes the output waveform response to a brief signal, i.e., an impulse. Thus, for a linear system, if the impulse response is known, it can be used to predict the response of the system to any given input. For the paired pulse input 410 and the output 430, FIG. 4C illustrates the impulse response 440.

FIG. 4D depicts the impulse response of FIG. 4C in the frequency domain, otherwise known as the transfer function 450. The transfer function 450 reveals a single dominant frequency (at approximately 0.1 on the x-axis in FIG. 4C). As can be appreciated from FIGS. 4C-D, looking at data in the frequency domain can make it easier to interpret (for example, viewing the data in the frequency domain may make changes between baseline and therapeutic characterizations more obvious or pronounced).

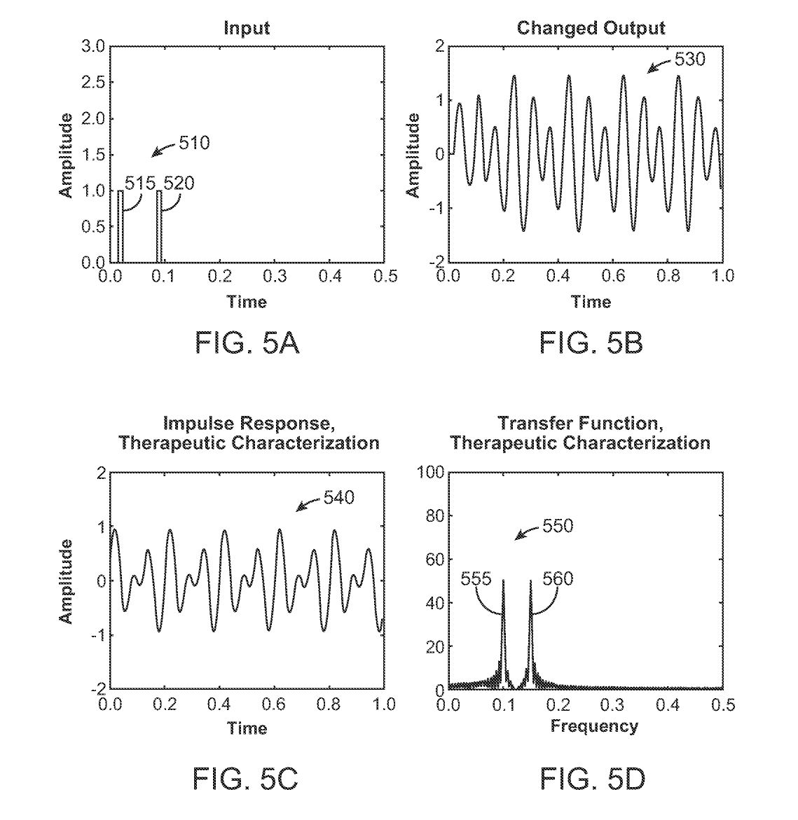

FIGS. 5A-5D graphically illustrate a possible therapeutic characterization for a neural system modeled as the linear system of FIGS. 4A-4D. The therapeutic characterization is undertaken when a change has been introduced to the neural system that is associated with a cell therapy. Introducing a change to the neural system 140 may constitute, for example, implanting stem cells into the brain, or implanting stem cells and then stimulating the region into which the cells have been implanted with electrical stimulation through an electrode connected to a pulse generator to encourage the cells to differentiate into a desired neural cell type. Obtaining a therapeutic characterization 150 may occur at one or more times relative to introducing the change 140, such as while the change is being introduced, immediately after the change is introduced, or some time after the change is introduced (e.g., minutes, hours, days, months, etc.).

FIG. 5A graphically illustrates a paired pulse input 510 used to obtain the therapeutic characterization that comprises individual pulses 515, 520. This paired pulse input 510 is substantially identical to the paired pulse input 410 (shown in FIG. 4A) that was used to obtain the baseline characterization of the linear system.

FIG. 5B shows the output 530 of the linear system based on the paired pulse input 510, and FIG. 5C shows the impulse response 540. Finally, FIG. 5D shows the transfer function 550 of the impulse response 540. In comparing the baseline characterization to the therapeutic characterization, it is apparent that the change that was introduced to the neural system had an effect. The change is apparent both in comparing the baseline characterization impulse response 440 to the therapeutic characterization impulse response 540 and in comparing the baseline characterization transfer function 450 to the therapeutic characterization transfer function 550. In particular, comparison of the therapeutic characterization transfer function 550 to the baseline characterization transfer function 450 reveals that the change introduced to the system results in two peaks (at approximately 0.1 and 0.15 in FIG. 5D) rather than the one peak (at approximately 0.1 in FIG. 4D) evident in the baseline characterization.

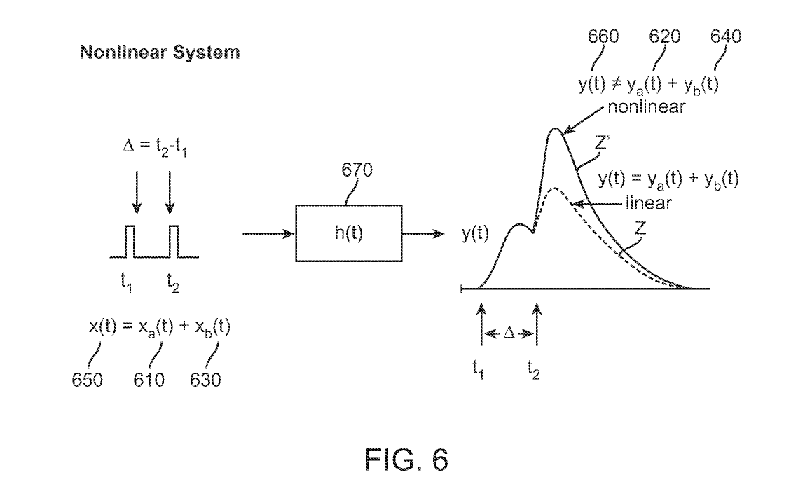

Nonlinear systems can be characterized by systems theory as well. Although the math is more complicated, a nonlinear model often will better approximate the actual behavior of the target than will a linear system model. Referring now to FIG. 6, an input stimulus xₐ(t) 610 generates a response yₐ(t) 620, and a stimulus x_{b}(t) 630 generates a response y_{b}(t) 640 where x_{b}(t)=xₐ(t+Δ) and y_{b}(t)=yₐ(t+Δ).

If this were a linear system, then a summation of the stimuli x(t)=xₐ(t)+x_{b}(t), would result in a summation of the two responses y(t)=yₐ(t)+y_{b}(t) according to the principle of superposition. Superposition states that the net response caused by two or more stimuli is the sum of the responses which would have been caused by each stimulus individually. In a nonlinear system, however, the two responses yₐ(t) 620 and y_{b}(t) 640 would not be simply superposed. Rather, in the nonlinear case, the output response may vary due to the nonlinear interaction between the input stimuli. For example, in FIG. 6, the dotted line Z shows what the output would look like if the system were linear, and the solid line Z′ shows a nonlinear output response. Of course, the nonlinear output response to a given input may vary from that shown in the example of FIG. 6 (e.g., the interaction of the two input pulses may cause a decrease in the amplitude of the output signal rather than an increase).

In the nonlinear case, the characterization requires nonlinear mapping between the input x(t) 650 and the output y(t) 660 and can be described by the kernels [h₀,h₁,h₂, . . . ] of a Volterra series. The discrete Volterra series representation for such a system, h 670, would be:

$$y [ n ] = {h}_{0} + {\sum }_{0}^{M - 1} {h}_{1} [ m ] x [ n - {m}_{1} ] + {\sum }_{0}^{M - 1} {\sum }_{0}^{M - 1} {h}_{2} ( {m}_{1} , {m}_{2} ) x ( n - {m}_{1} ) x ( n - {m}_{2} ) + … ,$$

where x[n] denotes the input and hᵢ denotes the kernels. The kernels may be interpreted as n^{th} order impulse responses which describe the dynamics of a system at each order of nonlinearity. The first order kernel corresponds to an average response of the system, if the system were linear. The second order kernel reflects the contribution or influence of a preceding pulse at the time another (“current”) pulse is delivered. Similarly, the third order kernel describes the modulatory contribution of the two preceding impulses to the current pulse in a stimulus train. In the nonlinear case, knowledge of the kernels enables linear and nonlinear characterization of the system and allows prediction of what the output response of the system will be to any given input.

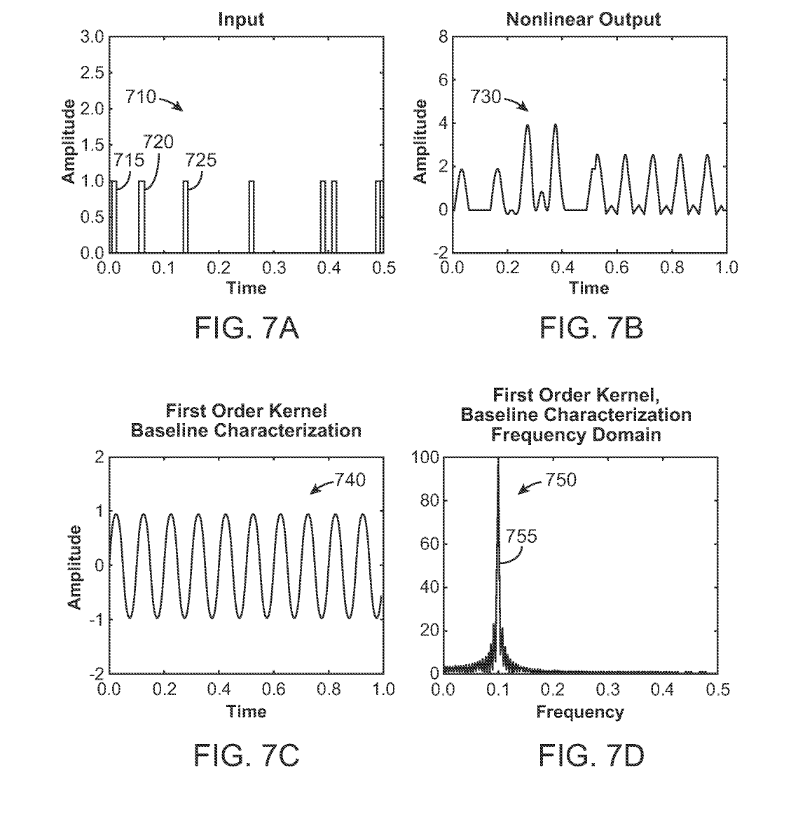

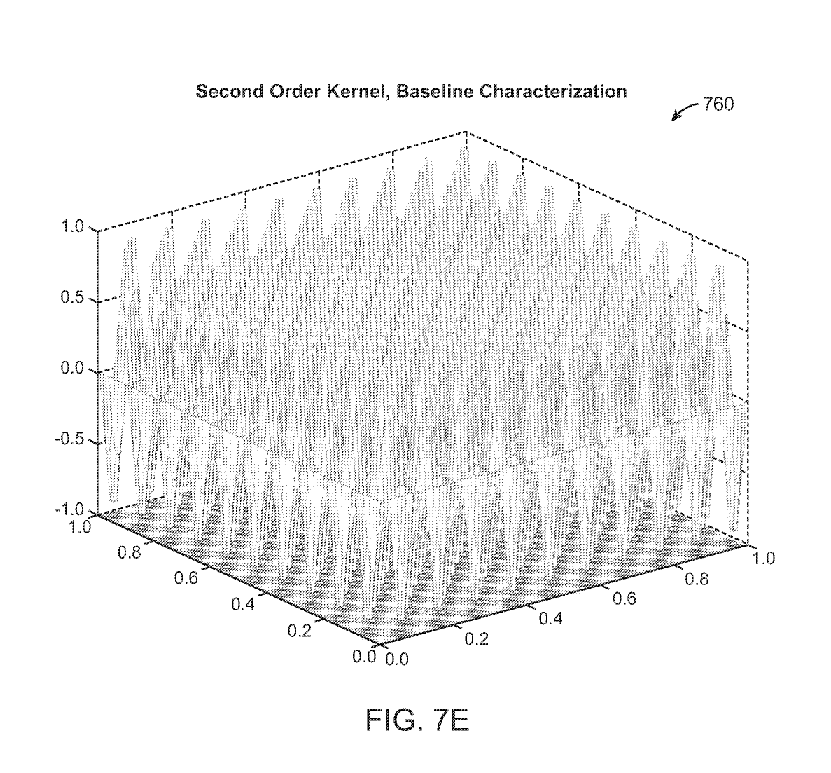

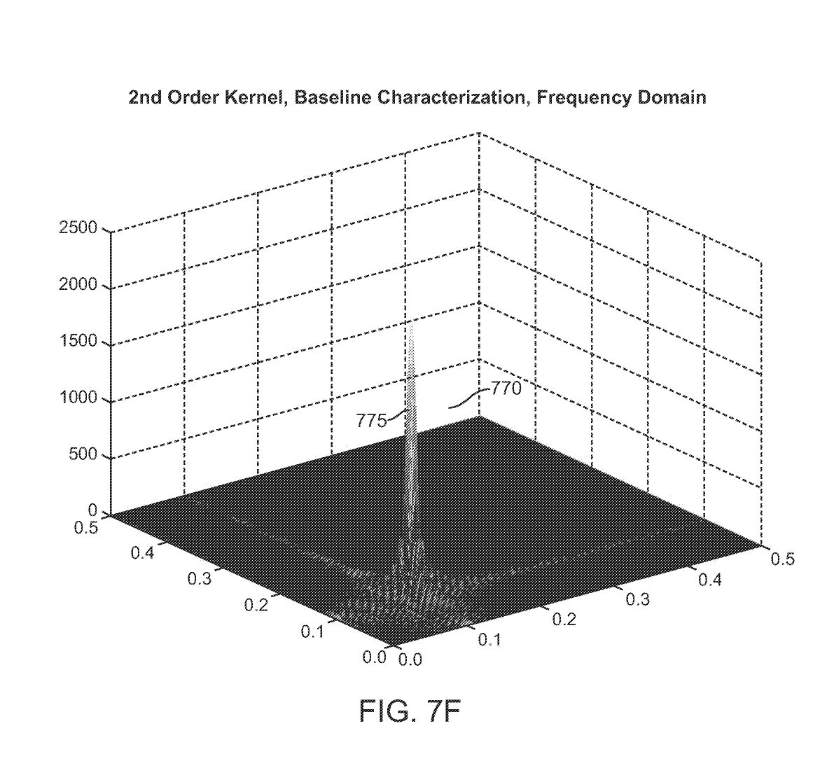

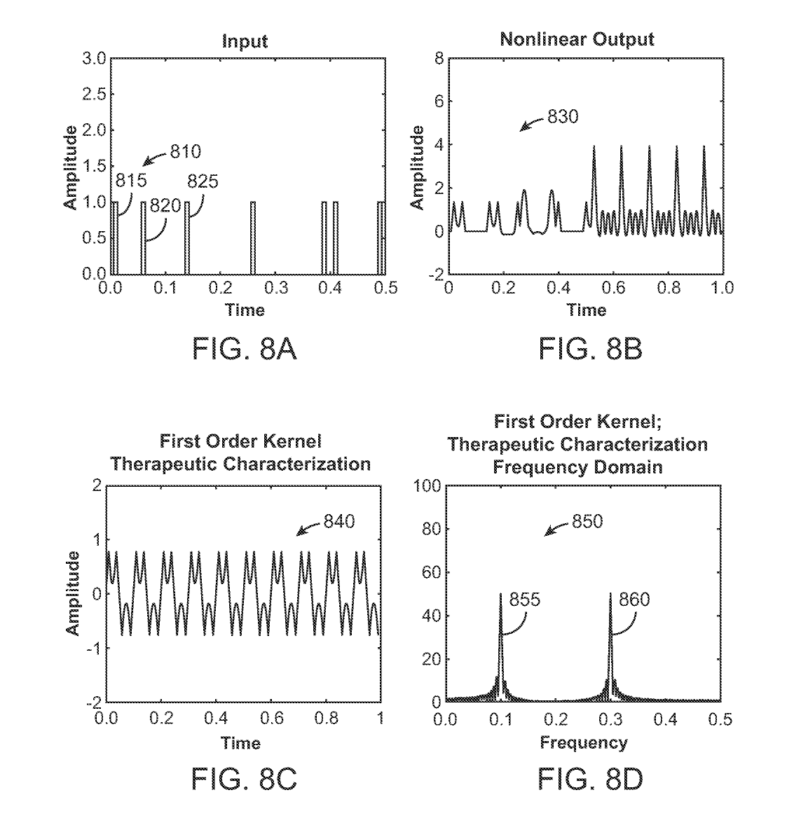

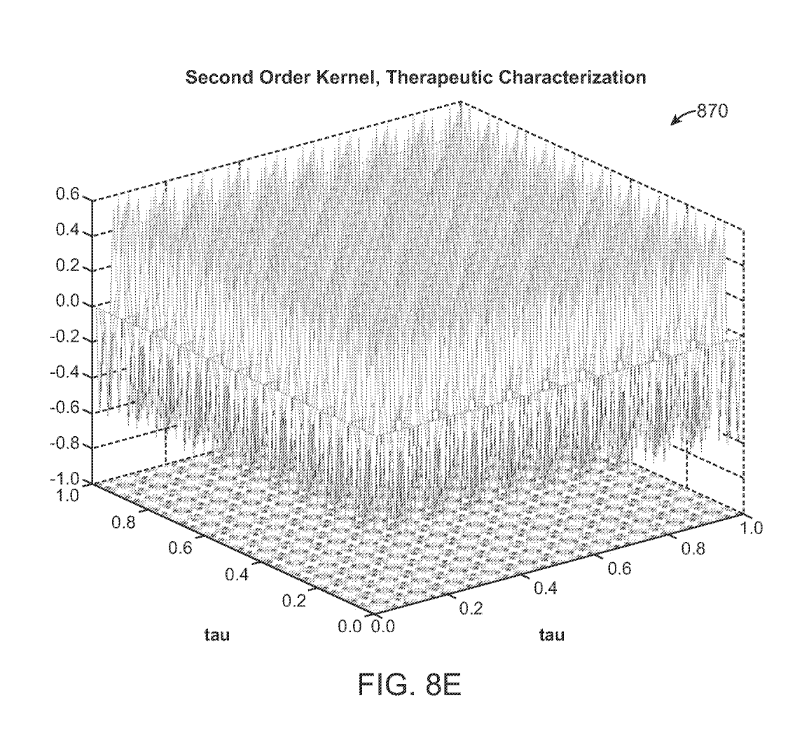

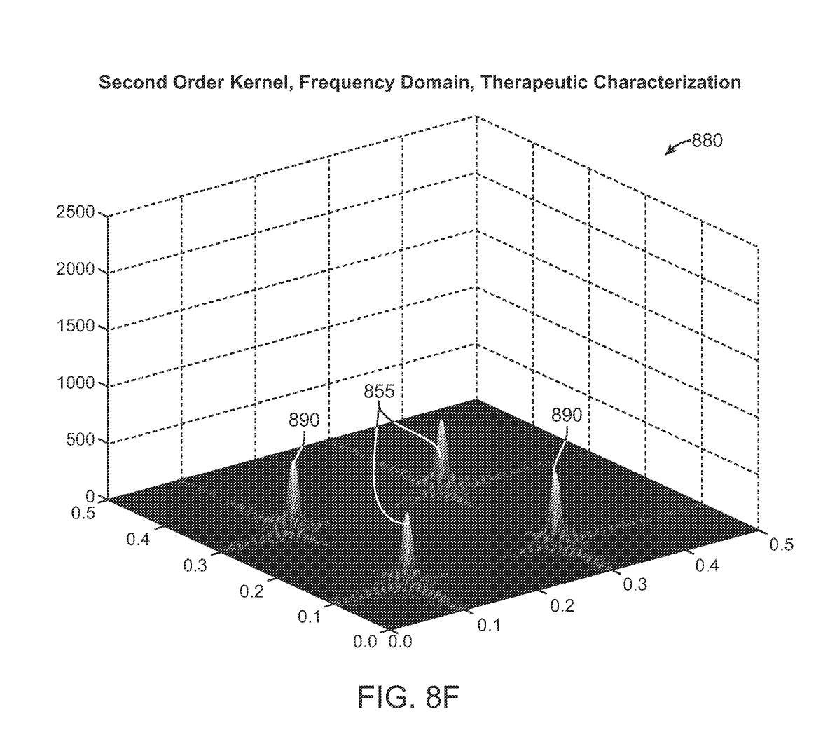

FIGS. 7A-7F and FIGS. 8A-8F illustrate baseline and therapeutic characterizations of a nonlinear system. In FIG. 7A, a segment of an input corresponding to a random pulse train 710 comprising individual pulses including first pulse 715, second pulse 720 and third pulse 725 is shown relative to amplitude on the y-axis and time on the x-axis. FIG. 7B illustrates an output response 730 that might be measured from a neural system based on the random pulse train input 710.

From the input and output, the n^{th} order kernels can be computed. FIG. 7C graphically illustrates a first order kernel 740 that can be computed from the output of FIG. 7B and the input of FIG. 7A using a Volterra series as described above. The first order kernel 740 can be extracted from the Volterra series and interpreted as an average response of the system if the system were linear. FIG. 7C illustrates the first order kernel 740 in the time domain. FIG. 7D alternatively illustrates the first order kernel in the frequency domain. In FIG. 7D, the frequency domain first order kernel 750 reveals a single dominant frequency 755 (at approximately 0.1).

FIG. 7E graphically illustrates a second order kernel 760 that can be computed from the output response (FIG. 7B) and the random pulse train input 710 (FIG. 7A). The second order kernel 760 can be extracted from the Volterra series and interpreted as reflecting the modulatory contribution of the preceding pulse to the current pulse, for example, the modulatory contribution of the first pulse 715 to the second pulse 720, the modulatory contribution of the second pulse 720 to the third pulse 725. A third order kernel extracted from the Volterra series would reflect the modulatory contribution of the preceding two pulses (the first pulse 715 and the second pulse 720 in the example) to the current pulse (the third pulse 725 in the example). FIG. 7E illustrates the second order kernel in the time domain (three dimensionally). FIG. 7F shows the second order kernel in the frequency domain. The frequency domain second order kernel 770 reveals nonlinearities at a single frequency 775 (approximately 0.1) indicating that preceding pulses occurring at a single frequency contribute to each current pulse (for example, the first pulse 715 in the random pulse train input 710 contributes to the second pulse 720). From this example, it will be apparent that knowledge of the kernels of a neural system not only enables the linear and nonlinear characterization of the system but also allows the prediction of the system output response to any given input.

FIGS. 8A-8F illustrate a possible therapeutic characterization of the nonlinear system for which a baseline characterization was described in connection with FIGS. 7A-7F. FIG. 8A shows a random pulse train input 810, including individual pulses 815, 820 and 825, that is substantially identical to the random pulse train input 710 used to obtain the baseline characterization for the nonlinear example. FIG. 8B illustrates an output response 830 based on the random pulse train input 810, FIG. 8C illustrates the first order kernel 840 in the time domain, FIG. 8D shows the frequency domain first order kernel 850, and FIGS. 8E and 8F show the time domain second order kernel 870 and frequency domain second order kernel 880, respectively.

In comparing the baseline and therapeutic characterization time domain and frequency domain first and second order kernels, it is apparent that the change introduced into the nonlinear system had an effect. For example, the frequency domain first order kernel 850 and frequency domain second order kernel 880 for the therapeutic characterization show peaks at approximately 0.1 and 0.3 (peaks 855 and 860 in FIG. 8D and peaks 885 and 890 in FIG. 8F), whereas only one peak at approximately 0.1 (peak 755 in FIG. 7D and peak 770 in FIG. 7F) in the frequency domain first order kernel 750 and frequency domain second order kernel 770 for the baseline characterization.

While the baseline and therapeutic characterizations of linear and nonlinear systems have been described above with respect to convolution equations and the Volterra series, it will be apparent to those skilled in the art that there are many alternate methods of characterizing a neural system including a target or targets to be monitored in connection with a cell therapy. For example, characterizations may be based on neural network theory, fuzzy logic, adaptive modeling techniques, pattern matching, wavelet theory, and data mining, as well as other methods.

An overall system including the target to be monitored can be modeled according to one of several possible system models, consistent with experimentally obtained information about the nature of the target or targets. For example, feedback, addition and cascade models have been used with different neural systems to describe behavior based on experimentally-observed data.

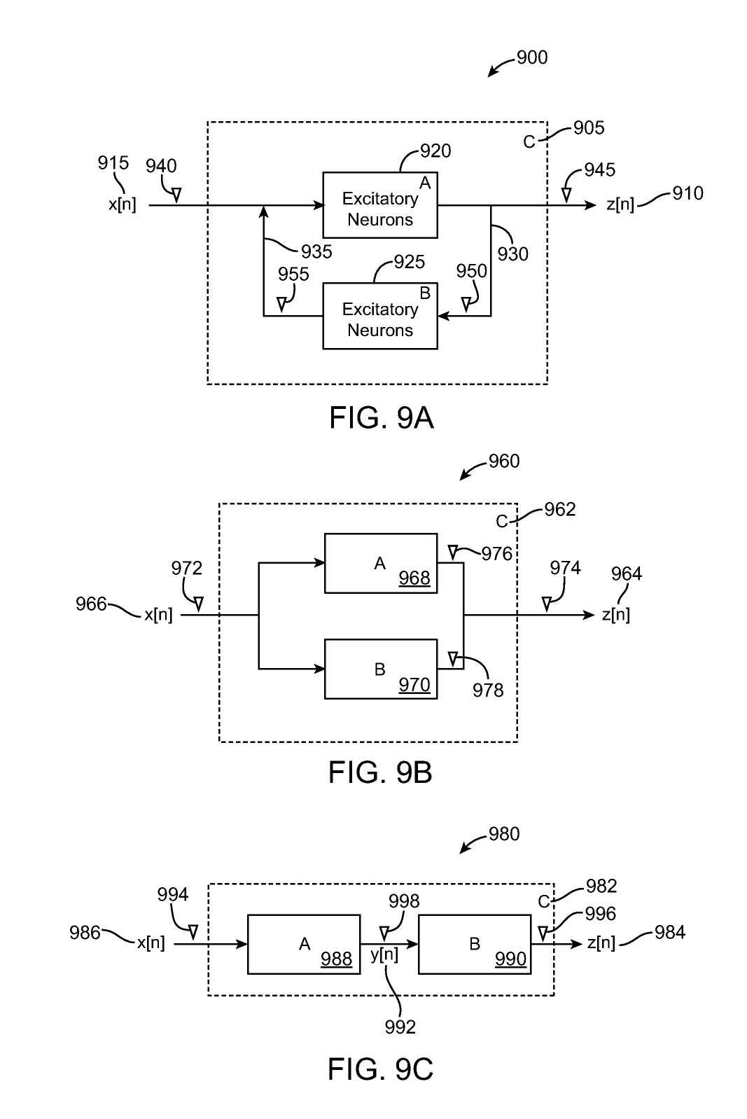

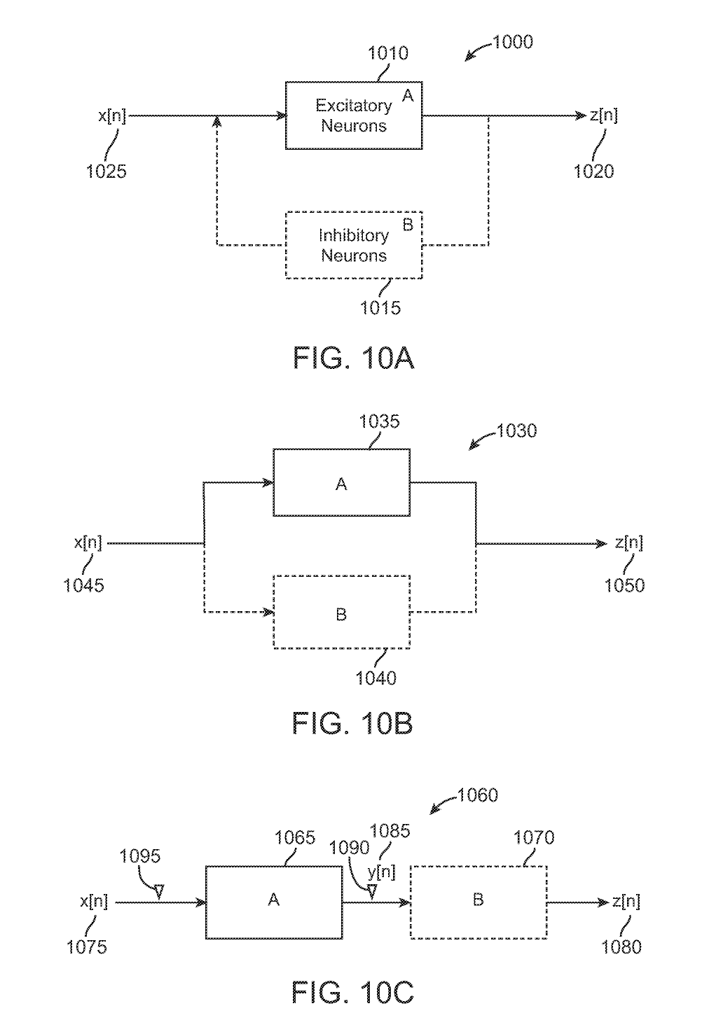

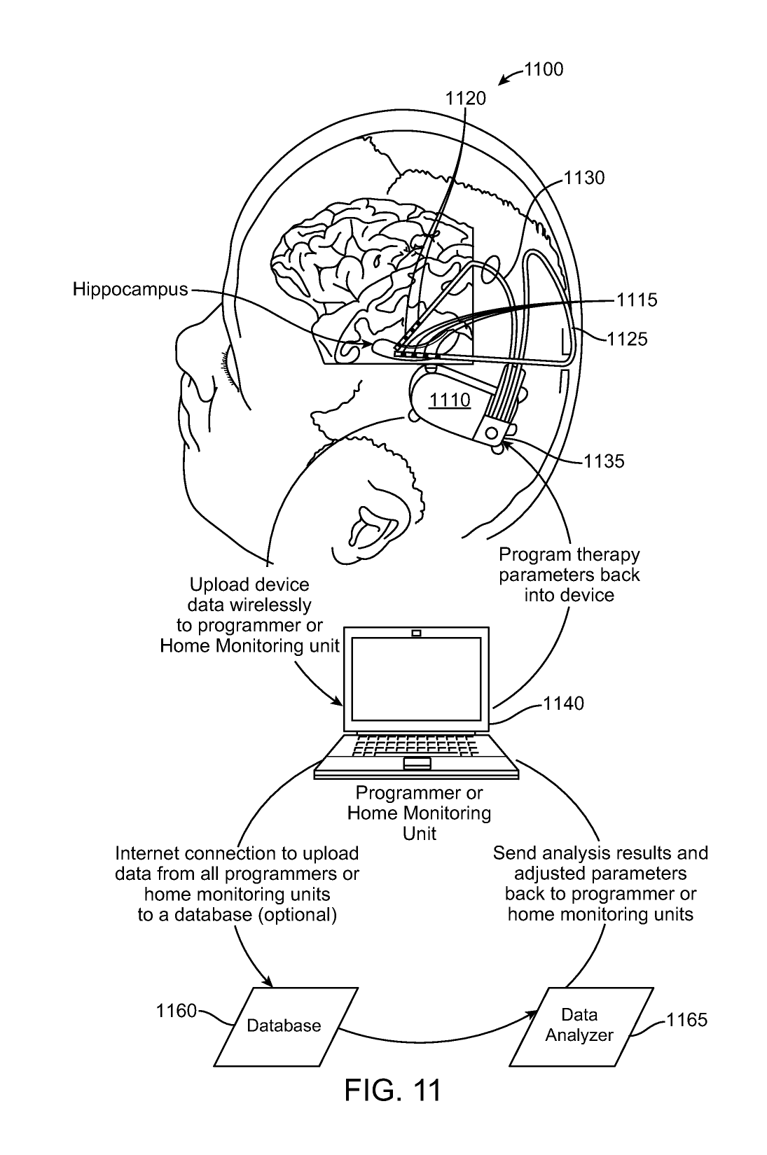

Referring now to FIGS. 9A-9C, some possible system models for neural systems are illustrated. FIG. 9A shows a feedback model 900 of an overall system “C” 905 with an input x[n] 915 and an output response z[n] 910. The overall system “C” 905 includes a first subsystem “A” 920 and a second subsystem “B” 925. Subsystem “A”920 is comprised of a first population of excitatory neurons and subsystem “B” 925 is comprised of a second population of excitatory neurons, but it will be appreciated that each subsystem may be comprised of the same or different cell types. For example, subsystem “A” 920 and subsystem “B” 925 may be composed of a population of excitatory neurons and inhibitory neurons, respectively, as is the case with the example of FIG. 10A. Alternatively, each subsystem might correspond to a node in a neural pathway, rather than to a physical population of neurons.

In the feedback model, the output 930 of subsystem “A” 920 is fed back as the input to subsystem “B” 925, and the output 935 of subsystem “B” is fed back to the input to subsystem “A” 920. (Of course, in a simplified feedback model, there might be no subsystems, and the output of a single system may be fed back as an input to the system).

To obtain characterizations of a neural system corresponding to a feedback model, the overall system can be characterized from the output, z[n] 910 of the overall system 905 relative to its input x[n] 915, and each subsystem (if any) may be characterized with respect to the output and input of each subsystem. For example, for a neural system corresponding to the feedback model shown in FIG. 9A, the input x[n] 915 may be an electrical stimulation pulse train, and a stimulating electrode 940 may be positioned in the anatomy at a location corresponding to the input x[n] 915. Recording electrodes 945, 950, and 955 (i.e., electrodes configured to sense the response of the neural tissue to the input x[n]), may be positioned in the neuroanatomy at locations corresponding to (1) the overall system output z[n] 910; (2) the output of subsystem “A”/input to subsystem “B” (for example, the recording electrode 950 might be physically placed at or near the population of neurons that constitutes subsystem “B” 925); and (3) the output of subsystem “B” 925 (recorded at recording electrode 955), which output is fed back as one input to subsystem “A” 920.

Thus, the I/O characterizations of the overall system (output z[n]/input x[n]) can be derived from the stimulation input at the stimulation electrode 940 and the output response recorded at the electrode 945; the I/O characterizations of subsystem “A” 920 can be derived from the input to subsystem “A”, namely, the stimulation input at the stimulation electrode 940, and the output responses recorded at the electrode 945; and the I/O characterizations of subsystem “B” 925 can be derived from whatever is recorded at the electrode 950 (input to subsystem “B”) and whatever is recorded at the electrode 955 (output of subsystem “B”).

Referring now to FIG. 9B, an addition model 960 for a neural system is shown. There are two subsystems shown in this example, although it will be appreciated that an addition model may be used with more than two subsystems. The overall system 962 has an output z[n] 964 and an input x[n] 966. Subsystem “A” 968 and subsystem “B” 970 are included as part of the overall system 962. The two subsystems share an input, namely, overall system input x[n] 966, and the output of subsystem “A” and the output of subsystem “B” are added together to produce the output response z[n] 964.

To obtain characterizations of a neural system corresponding to an addition model, the overall system can be characterized from the output, z[n] 964 of the overall system 962 relative to its input x[n] 966, and each subsystem may be characterized with respect to the output and input to each subsystem. For example, for a neural system corresponding to the addition model shown in FIG. 9B, the input x[n] 966 may be an electrical stimulation pulse train, and a stimulating electrode 972 may be positioned in the anatomy at a location corresponding to the input x[n] 966. Recording electrodes 974, 976, and 978 may be positioned in the neuroanatomy at locations corresponding to: (1) the output z[n] of the overall system; (2) the output of subsystem “A”; and (3) the output of subsystem “B.”

The I/O characterization of the overall system can be based on the input at electrode 972 and the output response recorded at electrode 974, and the I/O characterizations of subsystem “A” 968 and subsystem “B” 970 can be based on the input at the electrode 972 and the response recorded at the output of subsystem “A” (at the electrode 976) and the response recorded at the output of subsystem “B” (at the electrode 978), respectively.

Referring now to FIG. 9C, a cascade model 980 for a neural system is shown. There are two subsystems shown in this example, although it will be appreciated that a cascade model may be used with more than two subsystems. The overall system 982 has an output z[n] 984 and an input x[n] 986. Subsystem “A” 988 and subsystem “B” 990 are included as part of the overall system 982. The input 986 to the overall system 982 is the input to subsystem “A” 988, and the output y[n] 992 of subsystem “A” 988 is the input to subsystem “B” 990. An electrical stimulation input x[n] 986 for the characterizations is delivered at stimulation electrode 994, and a recording electrode 996 positioned at a location corresponding to the overall system output z[n] and the output of subsystem “B”, and a recording electrode 998 positioned at a location corresponding to y[n], the output of subsystem “A”/input to subsystem “B”, respectively, can be used to obtain characterizations of the overall subsystem and each subsystem.

If a model such as one of the models shown in FIGS. 9A-9C is used, then baseline and therapeutic characterizations of the overall system can be compared and characterizations of the subsystems can be compared to determine whether the behavior of the system/subsystems changes when a change associated with a cell therapy is introduced. (Alternatively or additionally, different therapeutic characterizations of the neural system can be compared, for example, therapeutic characterizations acquired at different times relative to delivery of a cell therapy.)

In some cases, it may be impractical to directly measure the output of a subsystem in a given systems model of a neural system. This may be due to the nature of the neuroanatomy and/or the nature of the cell type that comprises the subsystem. For example, the output of a subsystem may correspond to a physical location in the brain at which it is difficult to position a recording electrode. In another example, the subsystem may correspond to a population of inhibitory neurons such as interneurons. A group of interneurons may be defined as a single population but nevertheless may be widely dispersed in the neuroanatomy among other neural cell types (e.g., excitatory cells) that are not intended to be included in the subsystem. Thus, the physical area that a type of cell occupies may render it more difficult to use a single electrode to record an output of a subsystem comprising that cell type than it would if the cell type was characterized by a more dense structure.

Similarly, for various reasons, it may be contraindicated to position an electrode for delivering a stimulation input to a particular location in the neuroanatomy that corresponds to the input of a subsystem in a system model of a neural system. The design of an electrode, the materials available for implementing the electrode and/or coating it, and the nature of the stimulation to be delivered through the electrode each may have associated consequences. For example, consequences of electrode stimulation at a particular electrode-to-tissue interface may include scarring of the neuron's protective layer (glia and microglia). An electrode may become encapsulated by glial cells and therefore insulated from the neurons it is intended to stimulate, such that the flow of electrons is hindered and the impedance through which the stimulation is being delivered is undesirably increased. This can extend the effective distance between the electrode and the neurons that are intended to be stimulated, thus decreasing confidence that the stimulation delivered is the stimulation being input into the modeled system. The possible consequences associated with use of an electrode for delivering electrical stimulation may militate against its use in certain locations in the brain. (Improved electrodes may alleviate some of these consequences. Research is ongoing that is directed to improving the biocompatibility of stimulating electrodes which may lead to expansion of the locations at which a stimulating electrode effectively can be placed in the neural tissue in the future.)

There are other reasons that will be apparent to those skilled in the art why a recording electrode for sensing an electrophysiological response (either to serve as an input to a neural system (e.g., when the electrophysiological response corresponds to the result of some clinical test), or as an output of a neural system) or a stimulating electrode cannot or should not be placed in a particular location in the CNS. In such cases, a subsystem that is difficult to characterize directly with recording (or stimulation) electrodes, may be characterized indirectly. For example, the behavior of a subsystem “B” may be inferred from an I/O characterization of an overall system that includes the subsystem “A” and subsystem B and an I/O characterization of only subsystem “A”. Thus, using I/O characterizations of the overall system and subsystem “A,” subsystem “B” can be mathematically characterized indirectly. Alternatively, isolation one or more subsystems from another subsystem or subsystems may be used to simplify a particular systems model.

One possible technique for isolating subsystems includes using a pharmacological agent to temporarily block the functioning of the neural cells in one subsystem from another or other subsystems. For example, a system may be comprised of a first subsystem comprised of a population of excitatory neurons and a second subsystem comprised of a population of inhibitory neurons. The first subsystem may be isolated from the second subsystem by blocking the function of the inhibitory neurons in some way. For example, a pharmacological agent may be introduced to the population of inhibitory neurons (the second subsystem) to prevent that group of neurons from inhibiting their target neurons. Such blocking might be accomplished by locally delivering a drug that prevents the inhibitory neurons from inhibiting their target post-synaptic neurons. Thus, for so long as the function of the inhibitory neurons is blocked, the first subsystem can be isolated from the second subsystem. A given subsystem also may be isolated using strategic placement of stimulation and recording electrodes, for example, in an overall system including a first and a second subsystem, a stimulation electrode placed at the input to the first subsystem and a recording electrode placed at the output of the first subsystem will isolate the first subsystem from the rest of the system.

FIGS. 10A-10C each illustrate a systems model for a neural system in which a subsystem has effectively removed from an overall system so that other parts of the system can be isolated from the subsystem.

In FIG. 10A, a feedback systems model 1000 is shown with a subsystem “A” 1010 comprised of a population of excitatory neurons and a subsystem “B” 1015 comprised of a population of inhibitory neurons. The subsystem “A” 1010 is isolated from the subsystem “B” 1015 for the baseline and therapeutic characterizations by some means, such as injecting a drug into the population of inhibitory neurons corresponding to subsystem “B” that acts as a benzodiazepine receptor antagonist (e.g. flumazenil which acts as an inhibitor which acts as an antagonist to the GABA_{A} receptor). While the inhibitory neurons are blocked, an I/O characterization of the system 1000 based on an output z[n] 1020 measured at the system output and an input x[n] 1025 delivered at the input to the system will represent an I/O characterization of just the subsystem “A” 1010.

In FIG. 10B, an addition systems model 1030 is shown with two subsystems, subsystem “A” 1035 and subsystem “B” 1040, with an overall system input x[n] 1045 and an overall system output z[n] 1050. The subsystem “B” 1040 is blocked from the overall system in some fashion, so that the I/O characterization of the system 1030 represents the I/O characterization of subsystem “A” 1035 for so long as subsystem “B” 1040 remains blocked.

In FIG. 10C, a cascade systems model 1060 is shown with two subsystems, subsystem “A” 1065 and subsystem “B” 1070. The subsystem “A” 1065 is isolated from the subsystem “B” 1070, for example, by placing a recording electrode 1090 in a location in the anatomy corresponding to the output y[n] 1085 of subsystem “A” and a stimulation electrode 1095 at a location corresponding to the input x[n] 1075. If an I/O characterization is obtained by delivering an input electrical stimulation pulse train at the electrode 1095 and measuring the response at the electrode 1090, then the I/O characterization will be of subsystem “A” 1065 without subsystem “B” 1070.

When a characterization technique and/or systems model has been selected with which to monitor a target, implanting or positioning any required stimulation electrodes and recording electrodes or other sensors can be accomplished with a variety of approaches. For example, some of the same techniques available for identifying a target to monitor also may be used to implant electrodes or other sensing or stimulation circuits at a desired location in the brain. One such technique might be microelectrode recording (MER) after imaging has been used to generally locate a target. A microelectrode records activity of single neurons, and can assist a neurosurgeon in precisely determining when the microelectrode has reached a particular structure in the brain. For example, a microelectrode may be routed through the tissue while the surgeon listen for sounds or watches for visual signals recorded by the microelectrode as it passes through different structures (e.g., the basal ganglia, the thalamus, the substanitia nigra (STN), globus pallidus (Gpi), or ventral intermediate nucleus (VIM). Stereotaxis, where locations in the brain are identified using an external three dimensional frame of reference based on the Cartesian coordinate system, can be used alone or together with some other appropriate technique to place stimulation electrodes and sensors. Other techniques for implanting electrodes and sensors at desired locations in the central nervous system will be apparent to those skilled in the art.

It is anticipated that the methods, systems and devices can be used to good effect with a wide variety of cell therapies, where the baseline and therapeutic characterizations are used to monitor whether a change expected to be associated with the therapy has occurred after a change to the monitored system has been introduced (see FIG. 1, introducing a change 140 to the neural system).

A variety of stem cells are envisioned for use with embodiments of the monitoring system including embryonic stem cells, exogenous adult stem cells (especially, NPCs, a form of adult stem cells), or endogenous adult stem cells. Each of these cell types may have different potencies or capacities to differentiate into other cells. For example, embryonic cells that are derived from a very early-stage human embryo can be totipotent (capacity to differentiate into all of the cells that comprise any tissue and a placenta (e.g., cloning)) or pluripotent (capacity to differentiate into all of the cells that comprise any tissue, excluding a placenta). Using embryonic stem cells also may be desirable because of their ability to self-renew and their ability to be maintained in culture for an extended period of time without differentiating. Adult neuroprogenitor cells (NPC) have a more limited capacity to differentiate into different cell types. For example, some NPCs are unipotent, meaning that these stem cells only have the capacity to differentiate into one cell type. Other NPCs are multipotent, with the capacity to differentiate into more than one different cell type. NPCs may be desirable to use in a cell therapy because they may be more widely available (or less controversial) than embryonic stem cell types, even though adult stem cells may be harder to grow/maintain in vitro. In some cases, a patient's own endogenous adult stem cells may be useful in a cell therapy (e.g., if endogenous stem cells are at or near the site/neural circuit to be treated with the therapy or can be encouraged to migrate to that area from another location in the patient's body).

A cell therapy may include implanting stem cells into the neural tissue with the hopes that some or all of the implanted cells will self-renew and/or differentiate into cells of a desired type. Cell therapies can be used to not only generate new neurons but also generate support cells of the myelin sheath which act to insulate nerve impulses, facilitating communication in the brain. Cell therapies have also been used to generate large quantities of dopaminergic neurons to treat Parkinson's disease. Dopaminergic neurons are neurons that use dopamine as its neurotransmitter to communicate information. Dopaminergic neurons have the capacity to be both excitatory and inhibitory depending on the receptor to which that released dopamine binds. Other cell therapies that may be beneficially used with the disclosed methods, systems and devices will be apparent to one with skill in the art. A cell therapy also may make use of stem cells that are already in the patient's body (for example, the therapy may include encouraging endogenous stem cells such as NPCs to migrate to the target or to a neural system including the target where they are intended to differentiate into a desired cell type).

Diagnostic techniques may be used to define a given cell therapy. For example, the type of cells that are damaged, not functioning or functioning abnormally may inform the decision as to whether to implant embroynic cells or NPCs harvested from another subject (e.g., based on the versatility of one cell type versus the other). An assessment of the degree to which a neural circuit or population of neurons has been damaged may be determinate of how many stem cells to implant or to use (e.g., the more the damage, the more cells may need to be implanted). Patient specific factors may also be relevant to the type of therapy selected. For instance, in older patients, endogenous stem cells may not divide or may be slow to differentiate, even when encouraged with a form of stimulation, so in these patients, it might be desirable to implant a more vigorous form of stem cell.

In addition to delivering stem cells to a particular site, a therapy may also involve encouraging stem cells to differentiate into a desired cell type or migrate through use of some form of stimulation (e.g., electrical, pharmacological, optical, magnetic, etc.). Where the system for monitoring the target uses stimulation and recording electrodes to undertake characterizations, the same system also beneficially may be used to deliver a form of stimulation to encourage cell differentiation and/or migration and differentiation, as described more fully below. A stem cell therapy further may include using a combination of stem cell marking techniques and imaging to track the location of stem cells before and after they differentiate and/or to identify the type of cell into which a stem cell has differentiated.

Real time measurements reflecting certain electrophysiological properties of the stem cells can be obtained to assess progress of the cell therapy. Such electrophysiological properties may include voltage and current measurements on a variety of scales from single ion channel proteins to whole organs that may include the implanted stem cells. Monitoring these electrophysiological properties can be useful in determining whether the cell therapy is effective or needs adjustment. Electrodes and/or sensors for facilitating a cell therapy, including but not limited to devices for acquiring real time measurements reflecting electrophysiological properties of the stem cells, may be different (in terms of kind and/or location) than electrodes and/or sensors used for undertaking I/O characterizations.

After a change associated with a selected cell therapy has been introduced to a neural system (see FIG. 1, introducing a change 140), and baseline and therapeutic characterizations have been obtained (see FIG. 1, obtaining a baseline characterization 130 and a therapeutic characterization 150), then the results of the characterizations can be compared in an effort to assess whether the cell therapy has had any effect on the target being monitored (see FIG. 1, comparing therapeutic and baseline characterizations 160). The process of interpreting the results of the comparison 170 may range from the relatively simple to the relatively complex. The more complex is the function of interpreting the results of the comparison 170, the more likely that at least some of the components for implementing the function will be physically located externally of the patient (rather than wholly implanted).

In a relatively simple example, interpreting the results of the comparison 170 may involve determining whether the change (if there is a change) corresponds to an entry in a look up table populated with discrete values or ranges that can be correlated in some fashion to the comparison results. If the change matches one of the discrete values or falls within one of the ranges, then some conclusion may be reached about whether the cell therapy has been effective at all, or has been effective but not substantially so, or has caused some undesirable result. Alternatively, interpreting the results of the comparison 170 may involve determining whether the change (if there is a change) meets or exceeds a certain predetermined or programmable threshold. Such threshold may be a dynamic threshold derived from moving averages of data obtained from the patient or from groups of patients or other trends). If a threshold has been met or exceeded, this similarly may lead to conclusions about the effectiveness of the therapy. If there has been no change, or in some embodiments, a change that does not correspond to a value or range on a look up table or to a threshold, then the system may continue to monitor the target 175 by obtaining therapeutic characterizations, comparing the therapeutic and baseline characterizations, and interpreting the results.

In other embodiments, the system for monitoring may include adjusting the cell therapy 180, such as by adjusting one or more parameters according to which the cell therapy is being delivered, in an effort to change the baseline-to-therapeutic comparison so that, when the results are interpreted, it can be concluded that a positive therapeutic outcome (or at least not an undesirable therapeutic outcome) has been achieved. A given cell therapy may correspond to a wide variety of parameters. In one example, if the cell therapy involved implanting stem cells and then delivering a form of stimulation to encourage the stem cells to differentiate into a desired cell type, then the parameters may include the parameters that control the stimulation: for electrical stimulation, the parameters may include pulse shape, pulse amplitude and frequency, and the interstimulus interval, the number and kind of pulses within a burst and the time between bursts (i.e., interburst interval).

By way of one example, if the therapeutic characterization is the same as the baseline characterization, it may be inferred that the delivered cell therapy was substantially ineffective. If the therapeutic characterization differs from the baseline characterization, an assessment can be made as to whether the cell therapy was effective, partially effective, or harmful in achieving the desired effect.

For example, if the comparison 160 results in a determination that the desired effect has been achieved, the cell therapy could be terminated because additional cell therapy would not be required. Alternately, the comparison 160 may cause one to infer that an adverse effect, such as growth of a teratoma, has resulted from the provision of the cell therapy. If one observes a therapeutic characterization corresponding to a zero response or exhibiting no peaks in the frequency domain, this may indicate an adverse effect. For example, the lack of response and/or lack of peaks (in the frequency domain) may indicate that something (like a teratoma) is blocking an output response to an input (e.g., preventing transduction of a viable response to a deterministic input). If an adverse effect is determined, an alert can be provided to a healthcare provider and/or the cell therapy can be adjusted to counteract the cause of the adverse effect.

In still other instances, the comparison 160 may cause one to infer that progress has been made with respect to the desired effect. Depending on the precise results of the comparison 160, the cell therapy may be adjusted 180. Alternately, the cell therapy may be continued if further progress using the same treatment is believed to be likely. Other inferences may be drawn as a result of the comparison between the baseline characterization and the therapeutic characterization.

Correlation or covaraince methods such as cross correlation or a cross correlogram can be used to compare the baseline characterization to the therapeutic characterization in the time domain.

The basic cross correlation method can be described as:

$$( f * g ) [ n ] {=}^{def} {\sum }_{m = - ∞}^{∞} {f}^{*} [ m ] g [ n + m ] .$$

where f* denotes the complex conjugate of f. The presence of peaks in the cross correlation graph, can indicate points at which the two functions are correlated thus indicating that the baseline characterization and therapeutic characterization (or two compared therapeutic characterizations) are similar. This would indicate that the prescribed therapy was not having an effect. If the cross correlation graph is relatively flat, then it may be concluded that the functions are not well correlated and there was a large change between the baseline characterization and therapeutic characterization. Thus, it may be inferred that the therapy was having a more dramatic effect.

In addition, the functions can be converted to the frequency domain to determine how the fundamental properties between the baseline characterization and the therapeutic characterization (or between two therapeutic characterizations) have changed, if at all. For instance, if the therapeutic characterization in the frequency domain exhibits a larger signal at a particular frequency compared to the corresponding baseline characterization (or to a previous therapeutic characterization) in the frequency domain, it could be deduced that the characteristics of the neurons in the region have changed in quantity or behavior. Similarly, if a therapeutic characterization in the frequency domain exhibits more peaks compared to the corresponding baseline characterization (or a previous therapeutic characterization) in the frequency domain, then the additional peaks may signify a new neuron type is present.

Other techniques and methods can be used in conjunction with the therapeutic/baseline characterizations to assess whether a particular cell therapy has been effective. For example, magnetic resonance spectroscopy (“MRS”) is an imaging technique that has been used to quantify the number of NPCs in a region and track their neurogenesis.

In some embodiments, stem cells may be “marked,” such as by inserting a “reporter gene” (i.e., a green fluorescent protein), into the stem cells so that they can be tracked. Reporter genes are known that can be used with an imaging technique, such as MRS, to track stem cells in the body before they differentiate (or “specialize”). Reporter genes are also known that image as one color while a stem cell remains undifferentiated and then change to a different color when the cells differentiate into a particular cell type. It is envisioned that both types of markers would be useful with the methods, systems and devices described herein.

The neurostimulator can be programmed to deliver varying types of stimulation for the purposes of cell differentiation, strengthening and affecting the connections between neurons, inhibiting abnormal neural connections, increasing neuroplasticity or the like. Different types of electrical stimulation may be well suited for inducing differentiation of particular types of neurons. For example, higher frequencies may be best suited for inducing differentiation of inhibitory neurons, whereas lower frequencies may be best suited for inducing differentiation of excitatory neurons.

In addition, the amplitude of an electrical stimulus can be varied to manage the effect on implanted stem cells or neural connections. For example, delivering cell therapy using stimuli having larger amplitudes may strengthen neural connections and/or possibly result in more prolific cell differentiation. However, if the stimulation is too strong, it may weaken neural connections if proximate inhibitory neurons are activated. The amplitude of stimulation further may be correlated to other phenomena that suggests that certain stimulation parameters should be carefully controlled, for example, it has been postulated that chronic high amplitude stimulation may contributed to depression as a result of the stimulation desensitizing the affected neurons to naturally occurring forms of stimulation. Generally, the amplitude of the stimulation for the cell therapy should be high enough to accomplish the purposes of a particular cell therapy (e.g., whether that purpose is to encourage sufficient cell differentiation, to strengthen neural connections or to inhibit neural connections (as for example in an abnormally functioning neural circuit), provided that the amplitude otherwise is considered to be within safe limits for the patient.

Other factors may impact the effectiveness of a given cell therapy. For example, the frequency with which the stimulation is delivered may be important insofar as higher frequencies may encourage differentiation into one cell type (e.g., inhibitory neurons) and lower frequencies may encourage differentiation into another cell type (e.g., excitatory neurons). By way of further example, the interval between bursts or doses of stimulation may affect the therapy insofar different therapeutic results may flow from stimulation that is applied a predetermined number of times for a predetermined period (e.g., where a patient is continuously stimulated for an hour) than from stimulation that is applied according to a schedule (periodically or intermittently) or randomly relative to specific time periods.

The number and kind of stimulation devices and/or stimulation electrodes used for a given cell therapy may be dependent, at least in part, on the nature of the therapy and the nature of the neuroanatomy to which the therapy is to be delivered. In some instances, placing multiple stimulation electrodes along a neural pathway may be useful in control the spatial distribution of stem cells while or once the stem cells differentiate. The strategic placement of recording electrodes along with the stimulation electrodes may permit acquisition of data that can be used (along with baseline-to-therapeutic characterizations or therapeutic-to-therapeutic characterizations) to track the progress of a given cell therapy (e.g., for quantifying differentiation).

For example, in an embodiment, a cell therapy may include implanting stem cells along or near a pathway between stimulation and recording electrodes in an experimentally observed neural pathway. A plurality of stimulation electrodes preferably is implanted in a portion of a pathway where propagation of impulses begins and one or more recording electrodes preferably are implanted at or near cell bodies of a pathway where the propagation of the impulses converges. When stimulation electrodes are placed at or near the site where propagation of impulses begin, it is more likely that the stimulation will alter transmembrane ion distribution, an important factor in the development process of stem cells. Thus, properly placed stimulation electrodes can increase the likelihood that the stem cells will differentiate into the desired cell type(s). Placing the recording electrodes as close as practicable to the system output will increase the accuracy of the measured system responses (e.g., accumulation of ionic densities of cells of interest).

Cell therapies may be facilitated with techniques that are complementary or alternate to electrical stimulation to encourage stem cells to differentiate. For example, it is known that certain connections between neurons can be affected by chemical stimulation. More particularly, even when a group of implanted stem cells differentiates into desired neurons, the axons (an axon is the long fiber of a neuron that conduct electrical impulses away from the neuron cell body) may not be long enough or short enough to synapse onto the cells with which they are supposed to connect. Thus, it may be desirable to supplement the therapy that is intended to cause differentiation with a therapy that is intended to cause axons (either in the newly-differentiated cells or in neurons present in the area prior to implantation of the stem cells) to elongate or retract, as the case may be. Chemical stimulation has been known to promote such elongation or retraction. Thus, a given cell therapy may beneficially combine electrical stimulation (e.g., to encourage implanted stem cells to differentiate) and chemical stimulation (e.g., to encourage strengthening of connections between neurons or to prevent differentiated cells from forming undesirable connections with certain neurons).

Use of chemical stimulation, of course, should always be moderated by possibility that excessive chemical stimulation also may have undesired effects, such as desensitizing the brain (e.g., as when patients receiving L-Dopa for treatment of Parkinson's disease can become desensitized to the chemical stimulation, where increasing amounts of the drug progressively are needed to have the same desired effect on the patient's symptoms (such as reducing tremor). Increased doses may increase side effects and eventually increasing the dose may cease to have any positive effect on the symptom being treated with it. By using a combination of forms of stimulation to encourage stem cell differentiation, building of connections between cells, and spatial dispersion, etc., the risks of any one form if used alone might be avoided or at least ameliorated.

A cell therapy the progress of which can be monitored according to embodiments described herein may include using a form of stimulation to inhibit connections between neurons which connections are causing a problem. For example, in autism and schizophrenia, it is suspected that neurons form abnormal connections that contribute to symptoms or causes of these disorders. To minimize the effect of these abnormal connections, stem cells might be strategically implanted and then encouraged to differentiate into neurons with an inhibitory function that block or otherwise modulate the abnormal connections.

In alternate embodiments, a cell therapy may include features that encourage stem cells to differentiate into a desired cell type, such as a feature intended to induce the body to produce cytokines, growth hormones, and growth factors, or a feature that includes introducing cytokines, growth hormones and/or growth factors into the body. In addition, a cell therapy may include a feature intended to introduce or encourage cell signaling molecules that guide the differentiation of stem cells into a desired type of cell. These features and others may assist the differentiating or differentiated cells in survival and growth. Such therapies can be used to encourage growth of a patient's own stem cells and endogenous repair mechanisms and allow the patient's body to cope with damage from disease or injury. Similarly, implantation of growth factors at injury sites of the brain can encourage stem cells to multiply, migrate and differentiate into mature neurons.

Referring now to FIG. 11, one embodiment of a system useful in both monitoring and facilitating a cell therapy is illustrated. The system 1100 may include several implantable and external components. More particularly, an implantable neurostimulator 1110 includes a programmable pulse generator (not shown in FIG. 11) for delivering a variety of electrical stimulation signals through deep brain electrodes (e.g., deep brain electrodes 1115 and 1120) which are located at the distal portions of a first deep brain lead 1125 and a second deep brain lead 1130, respectively.

In FIG. 11 two deep brain leads are shown, each bearing four electrodes. As well be apparent to those with skill in the art, more or less leads may be provided bearing more or less electrodes. Each electrode may be configurable either to deliver stimulation or record an electrophysiological response from the brain (e.g., an electrocorticographic signal (ECoG) or both. One or more of the electrodes on a lead may be unused in a particular application.

The first and second deep brain leads 1125, 1130 have insulated conductors extending through the lead bodies which permit electrical communication between the electrodes 1115 and 1120 when the leads 1125 and 1130 are connected to the neurostimulator 1110 through lead connector 1135. Other types of electrode-bearing leads may be used. For example, a cortical strip lead (not shown) typically is provided with a paddle-like structure at a distal end thereof on which one or more electrodes are disposed. The cortical strip lead is intended to be oriented so that the electrodes rest against a surface of the brain under the dura mater.

As noted above, each of the deep brain leads 1125 and 1130 may be provided with multiple electrodes, and each electrode may be configurable either to deliver electrical stimulation to the brain from the pulse generator or to sense electrical activity from the brain which may be recorded and/or stored in the neurostimulator 1110. (In other embodiments, the electrodes may be configured to communicate wirelessly with the neurostimulator and/or external components.)

The electrodes 1115 and 1120, leads 1125 and 1130, and neurostimulator 1110 may be used, with one or more external components with which the neurostimulator is in communication, to characterize a predetermined neural system to monitor a cell therapy and to facilitate the cell therapy itself.

One or more of the electrodes 1115 and 1120 may be positioned at a location corresponding to one or more inputs to a neural system that previously has been identified by diagnostic or experimental techniques (or both). The one or more electrodes 1115 and 1120 may be configured as stimulating electrodes, for example, by circuitry in the leads 1125 and 1130 or in the neurostimulator 1110 or both, to deliver a form of electrical stimulation as the input to the neural system for the purposes of acquiring baseline and therapeutic characterizations of the neural system. Other of the electrodes 1115 and 1120 may be positioned at a location corresponding to an output of the predetermined neural system and configured as recording electrodes, to sense an electrophysiological response (e.g., sense an ECoG) from the neural tissue. The neurostimulator 1110 can be configured to deliver the form of electrical stimulation for the characterizations and to record, at least temporarily, the responses sensed by the recording electrodes.

The neurostimulator 1110 may also have some signal processing capability, ranging from signal amplication or signal filtering to performing the signal processing necessary to calculate the I/O characterizations and to cause the results to be displayed to the user. Alternatively, some processing of the signals being delivered or sensed through the leads may be carried out by a combination of signal processing circuitry in the leads 1125 and 1130 and in the neurostimulator 1110.

The neurostimulator 1110 may include circuitry such as telemetry circuitry that permits bidirectional communication between the neurostimulator and external components. The external components may include one or more of the following: (1) a computer 1140 that is used to program the neurostimulator for the characterizations (or for some other purpose, such as delivering stimulation to a population of implanted stem cells in order to encourage the stem cells to differentiate); (2) a computer that can be used by a patient as a form of home monitoring unit and which can be configured to deliver alerts or warnings to the patient as well as reminders for clinic appointments; (3) a database 1160 in which system data, such as programmed settings, raw or processed signals delivered or sensed through the neurostimulator 1110 and other data specific to a particular patient or particular patient population (e.g., stroke victims) can be stored and from which system data can be manipulated; (4) a data analyzer 1165 that is in communication either directly or indirectly with the database, the programmer 1140 and the neurostimulator 1110 and which may be configured for a variety of functions, for example, to carry out comparisons of characterizations (either baseline-to-therapeutic or therapeutic-to-therapeutic), to perform all or a part of the calculations for a characterization, to select a systems model for a given neural system, to automatically determine parameters, such as stimulation parameters, based on the electrophysiological response the recording electrodes are detecting, for example, on a responsive or adaptive basis (or both); (5) a display, such as an interactive display, provided either as a stand alone component or integrated with one or more of the other external components, on which any desired system data can be made accessible to a user (e.g., characterization data, the results of comparisons of characterizations, the past, current or recommended programming settings for the neurostimulator or other components of the system (such as which electrode “montage” is used or might be used, data relating to facilitation of a cell therapy, etc.); and (6) a device with which a patient can override system programming and cause an action to take place, such as turning off stimulation or triggering the system to record an event or a condition. One or more of the system components may be accessible over the Internet or other network.

Other possible components and configurations of components will be apparent to one with skill in the art. For example, the system 1100 may be provided with additional components such as a catheter for permitting localized delivery of drugs (for example, to block the function of a subsystem in a neural system so that other subsystems can be isolated from the blocked subsystem).

One way in which the system 1100 can be used for facilitating the therapy itself will now be described. One or more of the electrodes 1115 and 1120 may be positioned so that they are co-located with the site of a quantity of stem cells for which stimulation delivered by the neurostimulator 1110 is intended to encourage differentiation.

Alternatively, one or more of the electrodes may be positioned so that they are in the vicinity of a node of a neural pathway at which delivered stimulation might be expected to encourage differentiation of stem cells implanted elsewhere.

In still other arrangements, some electrodes may configured to deliver stimulation (and/or record responses corresponding to electrical activity of the brain) and other electrodes may be configured only to record, for example, at a location in the neural tissue at which the response evoked by the stimulation is likely to be recordable.

The neurostimulator may be programmed to continuously monitor the status of the implant site or the portion of the neural circuit and to deliver stimulation intended to encourage stem cell differentiation an a periodic or continuous basis.

Alternatively, the neurostimulator may be programmed to deliver stimulation on a responsive basis, such as only when the neurostimulator detects certain “events” occurring in the monitored signals.

U.S. Pat. No. 6,016,449 to Fischell et al. for “System for the Treatment of Neurological Disorders” issued Jan. 18, 2000 and incorporated by reference in the entirety herein describes systems, devices and methods for responsive stimulation that may applied to the methods, systems and devices described herein.

U.S. Pat. No. 6,810,285 to Pless et al. for “Seizure Sensing and Detection Using an Implantable Device” issued Oct. 26, 2004 and incorporated by reference in the entirety herein describes methods, systems, and devices for detecting events, such as using multiple neurostimulator channels and half-wave, line length, and area detection tools, to decide when an event warranting a response has occurred that may applied to the methods, systems and devices described herein.

Further, U.S. Pat. Pub. No. 2006/0212093 to Pless et al. for “Differential Neurostimulation Therapy Driven By Physiological Context” published Sep. 21, 2006, which is hereby incorporated in the entirety herein, describes methods, systems and devices for automatically adapting a form of stimulation delivered to neural tissue by selecting parameters based on the characteristics or features of the monitored signal or detected event (e.g., timing, frequency, phase, etc.) that may be beneficially used with the methods, systems and devices described herein. A form of adaptive stimulation is also described in U.S. Pat. No. 7,110,820 to Tcheng et al. for “Responsive Electrical Stimulation for Movement Disorders,” issued Sep. 16, 2006 in which the parameters of subsequently delivered stimulation are adjusted based on the physiological system's response to a previously delivered stimulation. The methods, systems and devices described in U.S. Pat. No. 7,110,820 could also be used beneficially with the presently described methods, systems and devices.

Programmable and/or adjustable electrical stimulation parameters may include, for example, waveform amplitude and/or frequency, current to be delivered through the electrodes, pulse shape (e.g., biphasic or otherwise charge balanced), first pulse delay, a hyperpolarizing prepulse, time between bursts of pulses in a pulse train, etc.

The computer 1140 also may be used to receive, process and store data recorded from the electrodes 1115 and 1120 (e.g., brain electrical activity detected from the electrodes or neurostimulator status data, such as remaining battery life, integrity of the connection between the neurostimulator (such as via impedance measurements) and the leads and/or electrodes, etc.).

From the computer 1140, data can be uploaded to or downloaded from other components. For example, data corresponding to the stimulation provided by the neurostimulator 1110 or to the brain's response to the stimulation may be uploaded from the computer 1140 (which may be a laptop or personal data assistant (PDA) to the database 1160 where it can be permanently stored and/or sent to the data analyzer 1165 for further processing and analysis. In some embodiments, the system 1100 may be configured to adaptively respond to data corresponding to the brain's electrical activity after a burst of stimulation has been delivered, for example, to adjust the stimulation parameters to provide a different result (e.g., to encourage more stem cells to differentiate), or to synchronize the therapy to the detected response, or to match one or more parameters of the stimulation to one or more parameters of the detected response (e.g., delivery therapy at the same frequency and/or in the same phase as the detected electrical activity).

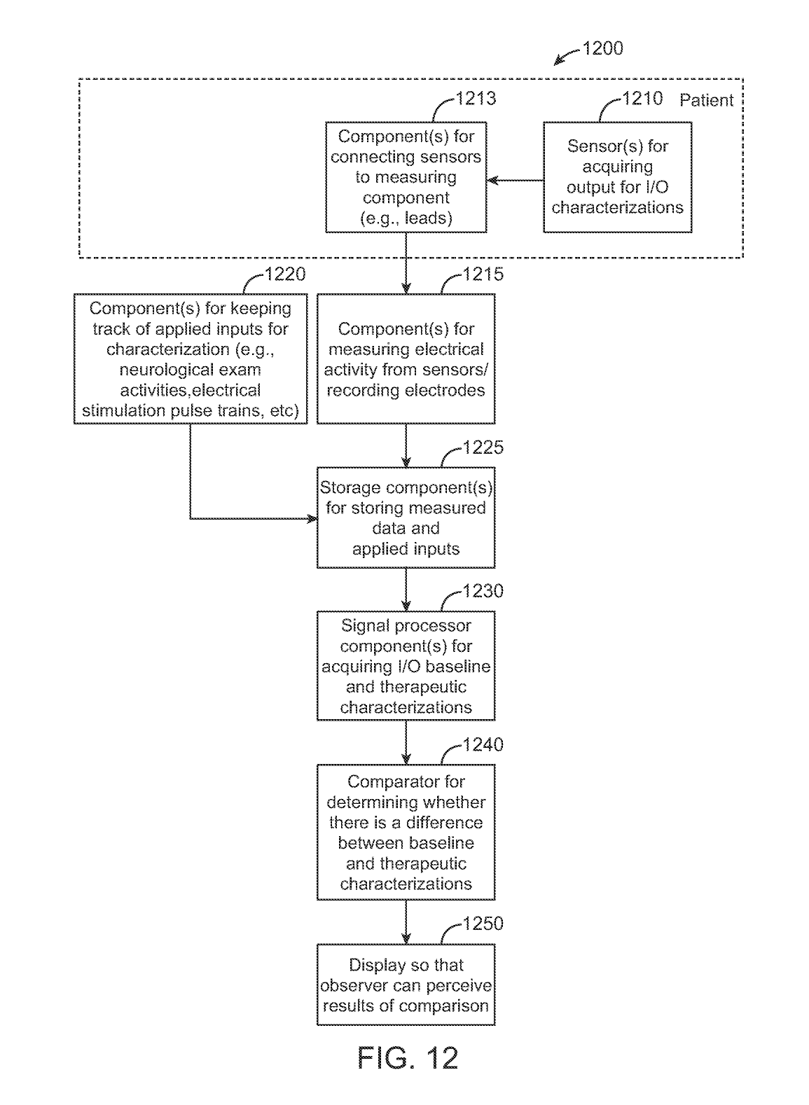

Referring now to FIG. 12, another embodiment of a system 1200 for monitoring a target in a neural system includes one or more sensors 1210 for acquiring electrical signals from the central nervous system (such as recording electrodes) and a measuring component 1215 for measuring the signals acquired from the sensor(s) through a component or components 1213 for permitting a connection between the sensor(s) 1210 and the measuring component 1215 (e.g., leads or a wireless connection). In one embodiment, a plurality of electrodes may be provided on the distal portion of either a cortical strip lead (designed so that electrodes will rest against a surface of the brain when the lead is implanted) or a deep brain lead (designed so that the electrodes will be implanted deep in the brain tissue). In this embodiment, the electrodes may comprise the sensors 1210 and the leads may comprise the connection 1213 between the sensors and the measuring component 1215.

The measuring component 1215 may be an implantable device that is configured to register the signals sensed from the sensors. The implantable device may also comprise a storage component 1225 for storing the measured sensor data and the applied inputs. The implantable device also may comprise all or some of the signal processor component(s) necessary for obtaining the I/O characterizations, based on information communicated to the signal processor component(s) 1230 from the storage component 1225.

Neurostimulation systems which include a neurostimulator that, in addition to being able to generate and deliver neurostimulation to the brain through cortical strip or deep brain electrodes connected to the neurostimulator via leads, also is capable of detecting, storing and accomplishing some signal processing on electrical signals measured from the electrodes via the leads (each electrode may be configured to either deliver stimulation or sense brain electrical activity) are described in, inter alia, U.S. Pat. No. 6,016,449 to Fischell et al. and U.S. Pat. No. 6,810,285 to Pless et al, both of which have been incorporated by reference in the entirety herein.

A component 1220 is provided for keeping track of the inputs delivered to the neural system for the characterizations. For example, in the case where electrophysiological responses recorded in response to clinical tests (e.g., instructions to the patient to move a limb or recite a phrase, or shining a light in a patients eyes, etc.) will be an input used in an I/O characterization of a neural system), the component 1220 may be configured to accept and store information corresponding to the type of clinical test, the time each test was delivered, and the electrode from which a response to the test was recorded.

By way of further example, where the inputs comprise a set of electrical stimulation pulse trains or a set of bursts of electrical stimulation, the tracking component 1220 may be configured to acquire and retain data corresponding what the parameters of the stimulation pulses or bursts are, and the times over which and the times at which the electrical stimulation is delivered to the patient.

Where the inputs comprise a combination of different input types, the tracking component 1220 may be configured to acquire and retain data corresponding to each of the different input types, such as instructions to the patient during a neurological examination (e.g., “try to raise your right arm,” “try to recite Table A from memory,” etc., electrical stimulation inputs, auditory inputs such as auditory sound waves, visual inputs such as light), and pharmacological inputs (e.g., used directly as inputs for I/O characterizations or used to isolate a subsystem in a systems model of a neural system, such as to block the function of a subsystem of inhibitory neurons).

The tracking component 1220 may be incorporated into an implantable device such as the neurostimulator described above. Alternatively, the tracking component 1220 may be comprised of some components that are external of the patient (e.g., a laptop computer for keeping track of the tests given in a neurological examination) and some components that are implemented in the implantable device.

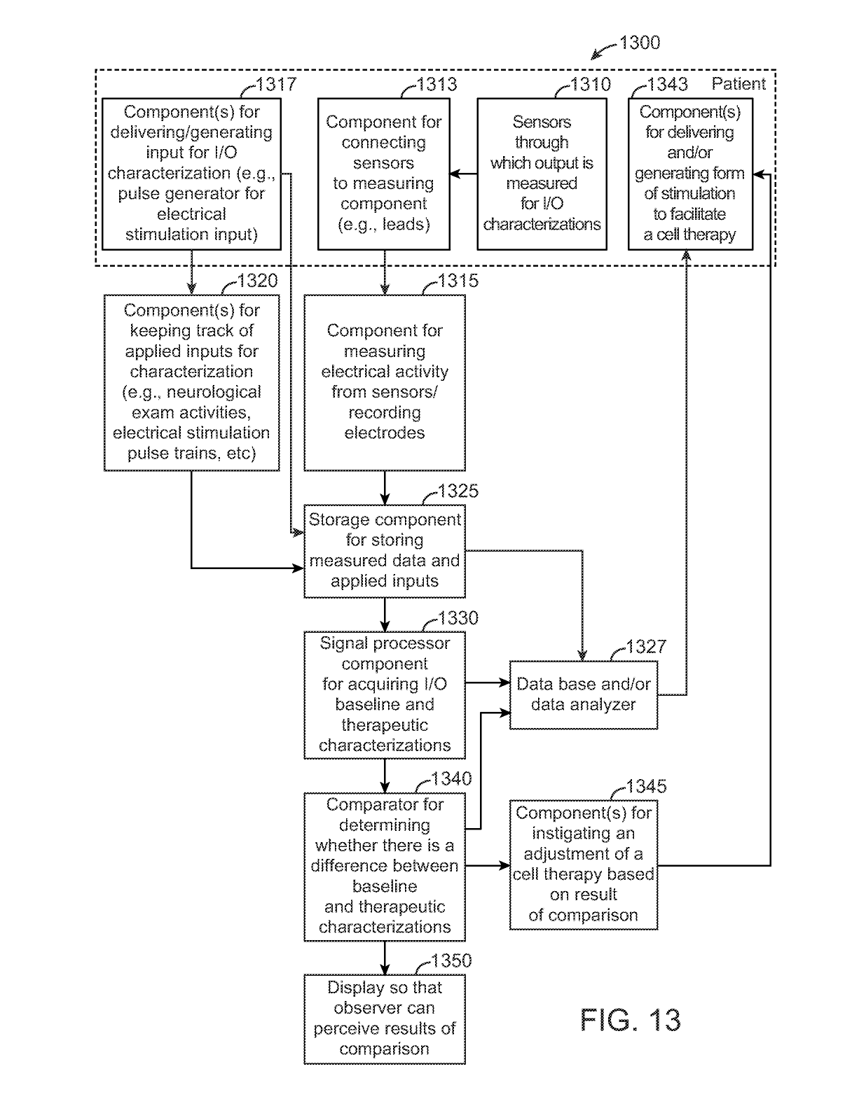

As will be apparent to those skilled in the art, one or more of the functional blocks described in connection with the diagram of FIG. 12 may be implemented with multiple components, multiple functions may be incorporated in a single component, and components may be designed to be implantable in a patient or to remain external to the patient when the system for monitoring a target is being used. For example, and with respect to signal processing, some initial processing of the sensed signals and applied inputs may be accomplished in an implantable component, for example, amplification, filtering, and low power operations that provide some insight into the nature of the signals being processed (see, e.g., U.S. Pat. No. 6,810,285 to Pless et al. which heretofore has been incorporated by reference, for a description of how half wave feature extraction, as well as line length and area-under-the-curve techniques can be used as low power techniques to detect characteristics in electrical activity sensed from a patient's brain). Additional processing may take place externally, by transmitting the data necessary to complete the I/O characterizations to external components, such as an external database and/or data analyzer (see, e.g., the functional block for database/data analyzer 1327 discussed further in connection with FIG. 13, below) which may be less constrained by power consumption limits (as might be the case with a battery-powered implantable device) and which may permit more complex computations and processing to be carried out on the data.

During or after the time that a cell therapy has been attempted, a comparator 1240 is used to compare baseline I/O characterizations to therapeutic characterizations (or to compare one therapeutic characterization to another therapeutic characterization). The functions carried about by the comparator may be implemented in an implantable device or partially in an implantable device and partially in external components with which the implantable device is selectively in communication, such as via telemetry or some other form of wireless communication.

A display 1250 is configured to display the results of the comparison in some manner, so that observers can perceive an indication of whether the cell therapy resulted in a change between different I/O characterizations.

Optionally, the system will have one or more additional capabilities, such as the capability to deliver one or more characterization inputs (for example, when a characterization input may comprise electrical stimulation, chemical or optical stimulation, etc.), the capability to generate one or more characterization inputs (e.g., electrical pulse trains from a pulse generator); the capability to long-term store any of, for example, the delivered inputs, measured outputs, I/O characterizations, comparison results; the capability of performing computationally intensive operations on the I/O data and comparison results; the capability to facilitate delivery of a cell therapy itself (e.g., by delivering electrical stimulation to encourage stem cells to differentiate into a desired cell type, to strengthen weakened connections between neurons, or to counteract abnormal connections between neurons); and the capability to responsively and/or adaptively change some aspect of a therapy based on the result of a comparison of a baseline and therapeutic characterization (or two different therapeutic characterizations), or the result of that comparison together with some additional information acquired about the progress of the therapy (e.g., from other strategically located physiological sensors, or the results of neurological testing in a clinical setting). Some of these optional features are illustrated in the block diagram of FIG. 13.

The system illustrated in FIG. 13 for monitoring and facilitating a cell therapy is provided with a component or components 1317 for delivering the inputs used in the I/O characterizations in addition to keeping track of them with the tracking component(s) 1320. For example, a component for delivering the inputs 1317 may deliver a form of electrical stimulation such as pulse train to be used as the inputs from a source external to the system, such as a pulse generator.

Alternatively, the component for delivering the inputs 1317 may be configured to deliver a form of chemical stimulation. Other possibilities include other forms of stimulation, such as optical, magnetic, auditory stimulation. In still other embodiments, the component(s) for delivering the inputs further may be configured to generate the inputs (or at least some of the inputs). For example, where one of the inputs for the I/O characterizations will be electrical stimulation, then a component for delivering and generating the inputs may be a programmable pulse generator with fully implantable components or a combination of implantable and external components.

In still other embodiments, the system for monitoring a target may further include components for generating one or more of the inputs used in the I/O characterizations, such as where an input comprises a form of electrical, chemical, or optical stimulation, etc. For example, a neurostimulation system with recording and/or detection capabilities as previously described above and in U.S. Pat. Nos. 6,016,449 and 6,810,285, may be have a pulse generator to generate electrical stimulation as inputs for the I/O characterizations with certain parameters (e.g., amplitude, frequency, interstimulus intervals, etc.) and at different times or on demand as communicated through an external command. The input-generating component 1317 may then deliver the stimulation to one or more of the electrodes through one or more leads of the neurostimulation system. The neurostimulator of the neurostimulation system (or some other system component) may have the capacity to configure each electrode either as a stimulation electrode or a sensing/recording electrode, as via switches.

The system for monitoring a target may have a database 1327 for long term storage of information, such as the inputs and outputs used in characterizations, the I/O characterizations themselves, and the results of comparisons of baseline and therapeutic characterizations. The database 1327 also may store details of the cell therapies attempted and any adjustments made to a given cell therapy that is monitored with the system of FIG. 13. Information on multiple patients with whom the system of monitoring a target is used may be collected in the database as well. Other useful data may be collected and retained in the database as will be apparent to one with skill in the art.

A data analyzer 1327 may be provided to operate on information in the database, such as the creation of statistical data concerning the results of particular cell therapies on particular patients or particular groups of patients. Where some of the signal processing for such things as sensed output responses, I/O characterizations, and comparison of baseline and therapeutic characterizations is relatively computationally intense and/or consumes a relatively large amount of power, some of the processing may be performed by a data analyzer implemented in external components. The functions performed by the data analyzer 1327 also may include sophisticated analyses of the I/O characterizations and of the results of the baseline-to-therapeutic comparisons (or therapeutic-to-therapeutic comparisons), where it may be impractical to include such analytic capacity in an implantable device given applicable technology limitations or operational constraints.

In still further embodiments, a system for monitoring a target may also include functional components for delivering or facilitating a cell therapy 1343. For example, a cell therapy may involve implanting stem cells into the neural system containing a target or targets to be monitored, and then applying a form of stimulation to the implanted cells (or in a pathway including the implanted cells) in order to encourage the cells to differentiate into a desired cell type. The system for monitoring and facilitating a cell therapy may also include components for delivering a pharmacological agent to designated areas of neural tissue to temporarily block the function of a group or type of neuron while characterizations are acquired.

A neurostimulation system such as that described above and in the U.S. Pat. Nos. 6,016,449 and 6,810,285 (previously incorporated by reference) may be configured to accomplish all of (1) providing sensors 1310 (e.g., electrodes) for detecting outputs for I/O characterizations; (2) providing components for connecting the sensors to a measuring component 1313 (e.g., leads bearing the electrodes at their distal ends and connectable to a neurostimulator at their proximal end); (3) providing measuring components 1315 for measuring the sensed electrical activity (e.g., a neurostimulator with recording and/or detection capability); (4) providing a storage component 1325 for storing data measured from the sensors and the applied inputs (e.g., a memory that receives data from the measuring component 1315, from any tracking components 1320 for keeping track of the applied inputs for the I/O characterizations, and from any components for delivering/generating inputs 1317); (5) providing a signal processor 1330 or some of the implantable components of a signal processor 1330 for acquiring I/O characterizations; and (6) providing a comparator 1340 for comparing the results of the baseline I/O characterizations to the therapeutic I/O characterizations.

The neurostimulator system can be interfaced with an external display or displays 1350 in order to provide feedback to observers about the results of the comparison or other status information about the system for monitoring a target. A display 1350 may be a computer monitor or PDA screen that communicates with the host computer and the implantable neurostimulator (and perhaps other implantable components) via an appropriate communications link, such as telemetry or telemetry with a modem or further wireless computer connection.

The neurostimulation system further can be configured with component(s) 1343 to provide a form of a cell therapy or to encourage a cell therapy (e.g., the neurostimulator can be configured to provide stimulation from a programmable pulse generator through certain electrodes on the electrode-bearing leads which stimulation is intended to encourage differentiation of implanted stem cells). As will be apparent to those skilled in the art, a single pulse generator in an implantable neurostimulator can be configured to selectively provide an electrical stimulation input for I/O characterizations and to selectively provide electrical stimulation for encouraging stem cell differentiation through the same or different electrodes. The neurostimulator and/or the leads may provide switching to allow certain electrodes to be used as stimulation electrodes (e.g., for providing inputs for I/O characterizations or stem cell therapy or both) in some circumstances and as sensors (e.g., as recording electrodes to measure outputs for I/O characterizations) in other circumstances. The neurostimulator may be configured to transmit information to components 1345 performing a database and/or data analyzing function concerning stimulation delivered to perform or facilitate a cell therapy so that it can be used in data analysis to lend meaning to, or further aid in the interpretation of, results of the comparison of baseline-to-therapeutic characterizations.

In still other embodiments, a neurostimulation system also can include a component or component(s) 1343 that will cause a cell therapy or stimulation intended to facilitate a cell therapy to automatically adjust based on the results of a baseline-to-therapeutic comparison or a therapeutic-to-therapeutic comparison. For example, if the results of a particular baseline-to-therapeutic characterization comparison suggests that the cell therapy has been too aggressive, then the results can be used to cause one or more parameters of the cell therapy to be adjusted in an effort to improve the therapeutic benefit (or to avoid undesirably results such as teratoma formation). Numerous parameters may be adjustable such as any of the pulse parameters (e.g., amplitude, frequency, pulse width, etc.) or the interstimulus intervals if electrical stimulation is being used to facilitate a cell therapy. The signal processing and decision-making involved in adjusting a cell therapy can vary from the simple (e.g., finding a comparison result in a look up table of possible options for therapy parameter adjustments and selecting the option that most closely matches the comparison result) to the complex (e.g., processing several channels of data together, such as channels of data reflecting the comparison results, the applied inputs and recorded outputs for the characterizations, data from other physiological sensors used in the cell therapy, and any stimulus applied to facilitate the cell therapy), and determining adjustments for cell therapy parameters based on computationally intense manipulations and/or interpretations of the data.

### Example

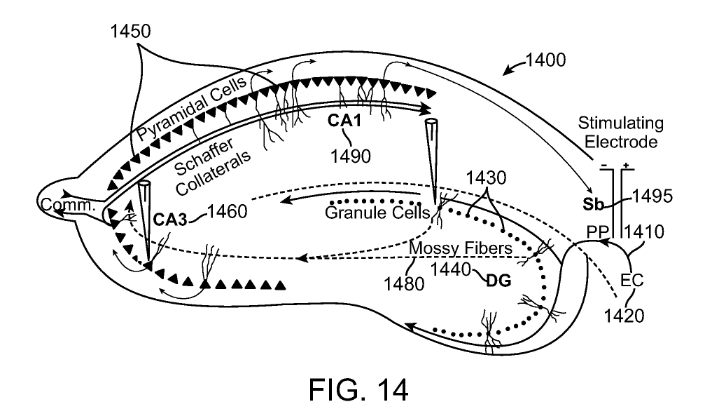

Referring now to FIGS. 14-19, examples of devices, systems and methods for monitoring a target in a neural system and, optionally, for facilitating a cell therapy, are described with reference to a brain hippocampal pathway, namely, the perforant path—dentate gyrus—CA3 region pathway.

The hippocampus is a paired structure in a normal human brain, which is to say that there are mirror image halves of the hippocampus physically located in the right and left hemispheres of the brain, inside the medial temporal lobes. The hippocampus is one of the structures that comprise the limbic system, which limbic system is believed to enable or control functions such as emotion, behavior, long term memory and olfaction. (Other structures in the limbic system include the amygdala, anterior thalamic nuclei and the cingulate cortex.)

The hippocampus is characterized by densely-packed layers of neurons, which is one reason why it has been used extensively in neurophysiological studies of the brain. Its function can affect inhibition, memory and spatial perception. Its abnormal function or other damage to the structure has been deemed a cause or source of disorders such as epilepsy and schizophrenia, Alzheimer's disease, amnesia and other conditions associated with aging process. Stroke (loss of blood supply to the brain) is another possible cause of damage to the hippocampus.

The hippocampus can be described in terms of portions that are substantially similar in composition but which form parts of different neural circuits.

The biggest source of input to the hippocampus is the entorhinal cortex (EC). This structure is also the biggest target of hippocampal output, which makes it a structure that is substantially and reciprocally connected with other parts of the cerebral cortex. The EC is the primary interface between the hippocampus and other parts of the brain.

In the hippocampus, the flow of information is largely in one direction, such that signals propagate through a succession of dense cell layers, namely, first the dentate gyrus (DG), then the CA3 layer, then the CA1 layer, then the subiculum (Sb), then out to the EC. There are other pathways in the hippocampus that output to other cortical areas, including the prefrontal cortex and the lateral septal area.

The pathway with which this example is concerned is one that involves the perforant path, the granule cells of the dentate gyrus, the pyramidal cells of the CA3 region and the pyramidal cells of the CA1 region and the subiculum (i.e., a perforant path—dentate gyrus—CA3 region pathway).

With reference to FIG. 14, several hippocampal neural connections or pathways 1400 are described with respect to a neural circuit model of the hippocampus. The perforant path (PP) 1410 is a major input to the hippocampus. Axons from the perforant path 1410 arise principally in the entorhinal cortex 1420 and project to the granule cells 1430 of the dentate gyrus (DG) 1440 and the pyramidal cells 1450 of the CA3 region 1460.

A mossy fiber pathway is defined by the mossy fibers 1480 which are the axons of the DG granule cells 1430, extend from the DG to the pyramidal cells 1450 of the CA3 region 1460 and thus form a major input to the pyramidal cells of the CA3 region. Multiple granule cells 1430 can synapse onto a single pyramidal cell 1450 of the CA3 region 1460.

Another pathway including the hippocampus is the Schaeffer Collateral/Associational Commissural Pathway, which starts with axons that project from the CA3 region 1460 to the CA1 region 1490. The axons projecting from the CA3 region 1460 may project from CA3 neurons in the same hippocampus or from CA3 neurons in the hippocampus in the other hemisphere of the brain. The fibers from the opposite or contralateral hemisphere are “commissural” because they cross from one hemisphere to the other.

Still another pathway including the hippocampus is the CA1-Subiculum-EC pathway, which is the principal output of the hippocampus. This pathway is not uni-directional, however. The first part of the pathway corresponds strictly to the anatomy insofar as the distal end of the CA1 region 1490 projects to the proximal end of the of the subiculum (Sb) 1495 (i.e., there is a connection between the cells nearest to the CA1-Sb junction). In the second part of the pathway, cells in the distal CA1/proximal Sb region project to the lateral EC 1420 and cells in the proximal CA1 region/distal Sb project to the medial EC 1420. (The input to these cells follows a similar pattern insofar as cells in the distal CA1 region/proximal Sb receive input from the lateral EC and cells in the proximal CA1 region/distal Sb receive input from the medial EC.)

A target to be monitored may be included in a neural system corresponding to one or more of the hippocampal pathways 1400. For example, diagnostic testing may lead to the conclusion that there has been damage to the hippocampal region in the pathway including the perforant path, the dentate gyrus, and the CA3 region because of a stroke or due to some other neurological disorder or condition.

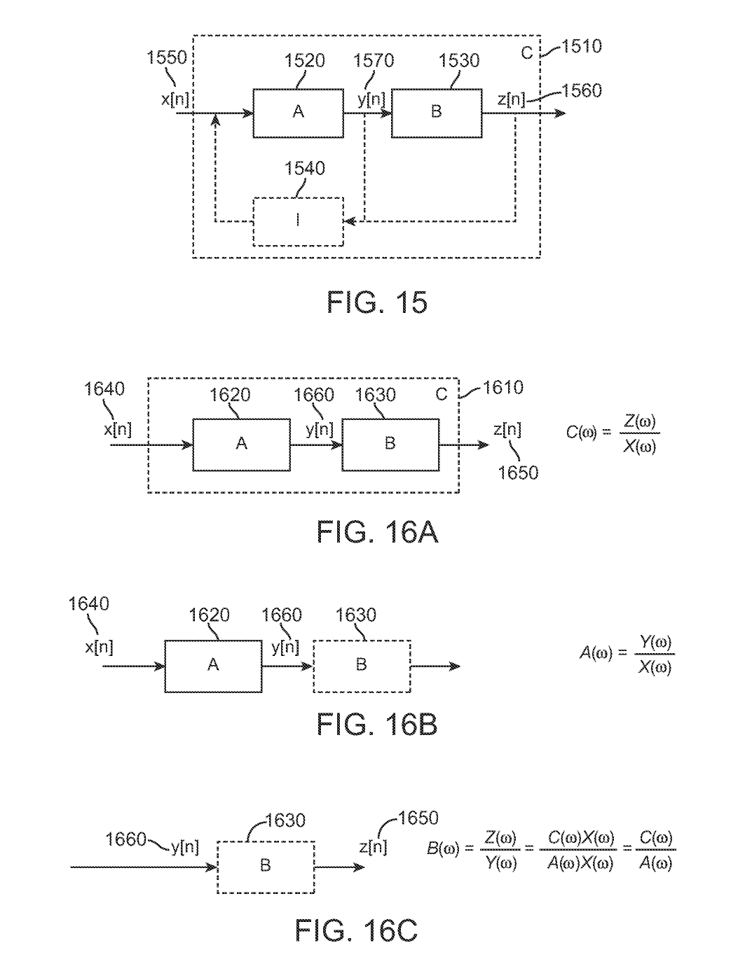

Systems models can be used to model the behavior of a neural system comprising a hippocampal pathway. For example, and referring now to FIG. 15, the perforant path 1410—dentate gyrus 1440—CA3 region 1460 pathway can be modeled as an overall system “C” 1510 with a subsystem “A” 1520, a subsystem “B” 1530 and a subsystem “I” 1540. The subsystem “A” 1520 corresponds to the granule cells 1430 of the dentate gyrus 1440. The subsystem “B” 1530 corresponds to the pyramidal cells 1450 of the CA3 region 1460. The subsystem “I” 1540 corresponds to a population of interneurons that are in communication with pyramidal cells and granule cells and function to inhibit the pyramidal cells and granule cells.

The input to the overall system “C” 1510 is the perforant path 1410 and corresponds to x[n] 1550 in FIG. 15. The output z[n] 1560 of the overall system “C” 1510 corresponds to the pyramidal cells 1450 of the CA3 region 1460 (which are an input to the CA1 region 1490).

The output y[n] 1570 of subsystem “A”1520 corresponds to the granule cells 1430 of the dentate gyrus 1440. As is shown in FIG. 15, the output y[n] 1570 also is an input to subsystem “B” 1530 and an input to subsystem “I” 1540.

As explained above, the interneurons comprising subsystem “I” 1540 have an inhibitory function. Anatomically, they are widely dispersed in the hippocampus and each cell occupies a relatively large area. Therefore, isolating this particular population of neurons in order to I/O characterize it as a subsystem may be impractical. For example, it may be a challenge to position a stimulating electrode so that it will deliver stimulation (as an input for an I/O characterization) so that the stimulation acts on the entire population of interneurons that constitutes subsystem “I” 1540. Similarly, it may be a challenge to locate a recording electrode or electrodes so that these electrodes will record the output of the subsystem “I” 1540 to the stimulation input and only that output.

In addition, the presence of the subsystem “I” 1540 in the systems model of the perforant path-dentate gyrus-CA3 region pathway complicates the systems model. That is, and referring now to FIG. 16A, if the subsystem “I” 1540 is eliminated, the systems model becomes a simple cascade model defined by an overall system “C′” 1610 with a subsystem “A” 1620 (granule cells 1430 of the dentate gyrus 1440) and a subsystem “B” 1630 (pyramidal cells 1450 of the CA3 region 1460). While the function of subsystem “I” is blocked, the blocked cells in that subsystem will not be functioning to inhibit their target neurons. The input x[n] 1640 to the overall system “C′” 1610 is the input to subsystem “A” 1620 (dentate gyrus 1440) and the output z[n] 1650 of the overall subsystem “C′” is also the output of the subsystem “B” 1630 (CA3 region 1460). The output y[n] 1660 is the output of the subsystem “A” 1620 and the input to subsystem “B” 1630.

The subsystem “I” 1540 may be eliminated from the overall system “C” 1510 during the time in which baseline and therapeutic I/O characterizations are acquired by, for example, temporarily blocking the function of the interneurons with a pharmacological agent that is designed to block only the inhibitory function and not the function of other neural cell types. Suitable agents may be a benzodiazepine receptor antagonist (e.g. flumazenil which acts as an antagonist to the GABA_{A} receptor on the interneurons). The drug may be delivered locally to the hippocampal region via a needle or catheter or using some other appropriate technique.

A simple linear I/O characterization for the overall system “C” 1610 (transfer function of the impulse response) can be described by:

$$C ( ω ) = \frac{Z ( ω )}{X ( ω )}$$

With reference to FIG. 16B, the transfer function for just subsystem “A” 1620 can be described by:

$$A ( ω ) = \frac{Y ( ω )}{X ( ω )}$$

Finally, and with reference to FIG. 16C, the transfer function for subsystem “B” 1630 can be derived from the transfer functions of the overall system “C” 1610 and the subsystem “A” 1620 according to:

$$B ( ω ) = \frac{Z ( ω )}{Y ( ω )} = \frac{C ( ω ) X ( ω )}{A ( ω ) X ( ω )} = \frac{C ( ω )}{A ( ω )}$$

This methodology can be extended for a cascade model that is nonlinear using a Volterra Series. Thus, instead of impulse responses, nth order kernels could be computed for the overall system “C,” subsystem “A” from the I/O data. The nth order kernels for subsystem B can be computed from mathematical derivations similar to the linear system equations describe above.

It should be noted that it may be appropriate, in some instances, to directly characterize each of the subsystems in a systems model together with the overall system, by isolating each subsystem with strategically placed stimulating and recording electrodes (to provide a characterization input and to record a characterization output, respectively). In the example of the hippocampus, placing a stimulating electrode at the input y[n] 1660 to subsystem “B” 1630 may be contraindicated because the mossy fibers 1480 that comprise this input are characterized by complex, varying physiology and using stimulating electrodes in the mossy fibers may not yield reliable or predictable results. Thus, indirectly characterizing subsystem “B” 1630 using the direct characterizations of subsystems “A” 1620 and the overall system “C” 1610 may be the best approach.

Accordingly, the above-described calculations can be used to obtain baseline I/O characterizations of the overall system “C′” 1610 and the subsystem “A” 1620 and subsystem “B” 1630 and one or more therapeutic characterizations of the overall system “C′” and subsystems “A” and “B” during or after the time a change has been introduced to the system (e.g., an implant of stem cells to the hippocampal region, or an implant of stem cells to the hippocampal region followed by delivery of a form of stimulation (e.g., electrical) intended to encourage the stem cells to differentiate into a desired cell type).

The target(s) in the neural systems corresponding to the systems model can be monitored by comparing the baseline characterization to one or more therapeutic characterizations. If there is a difference between the baseline characterization and a therapeutic characterization, then conclusions may be reached about the relative effectiveness of the therapy. In addition, comparison of past, present, and future therapeutic characterizations can also be used to aid in measuring the effectiveness of the cell therapy. The degree of the difference also may inform adjustments to the therapy to either make the difference greater or smaller or in an effort to alter the nature of the difference (e.g., change where the peaks are in the therapeutic characterization).

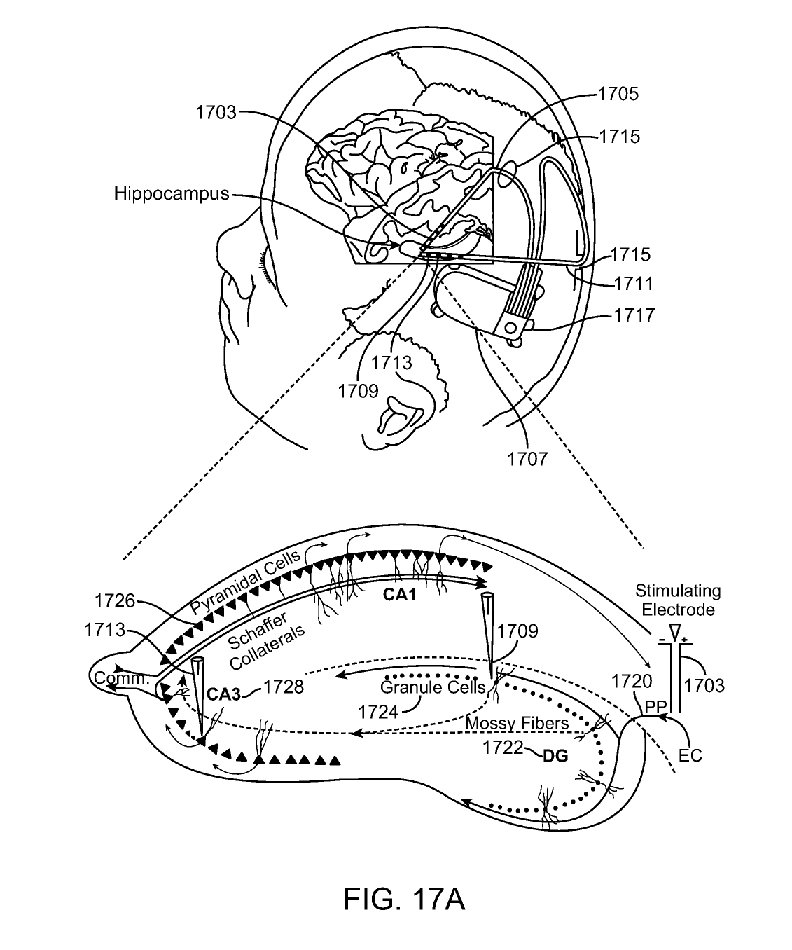

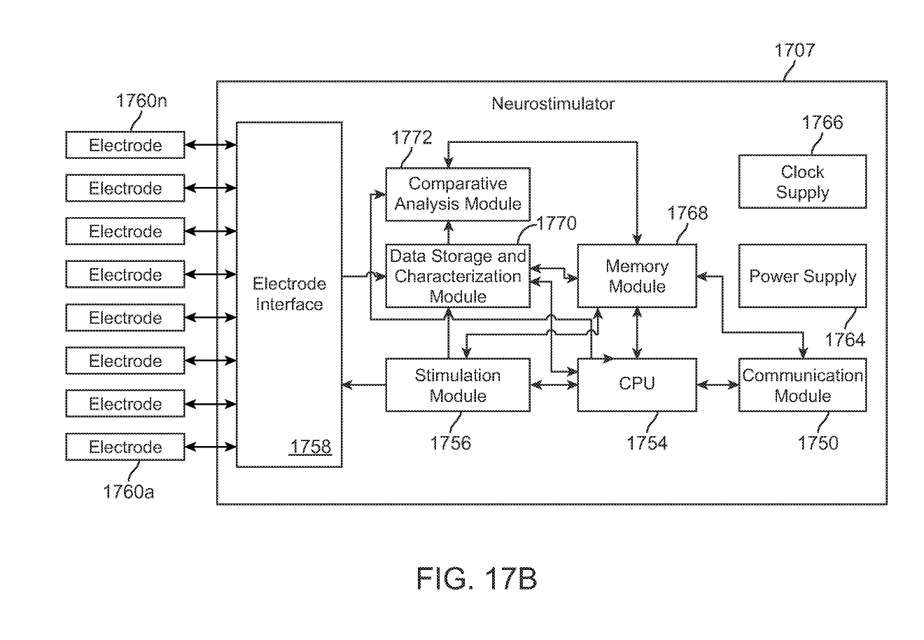

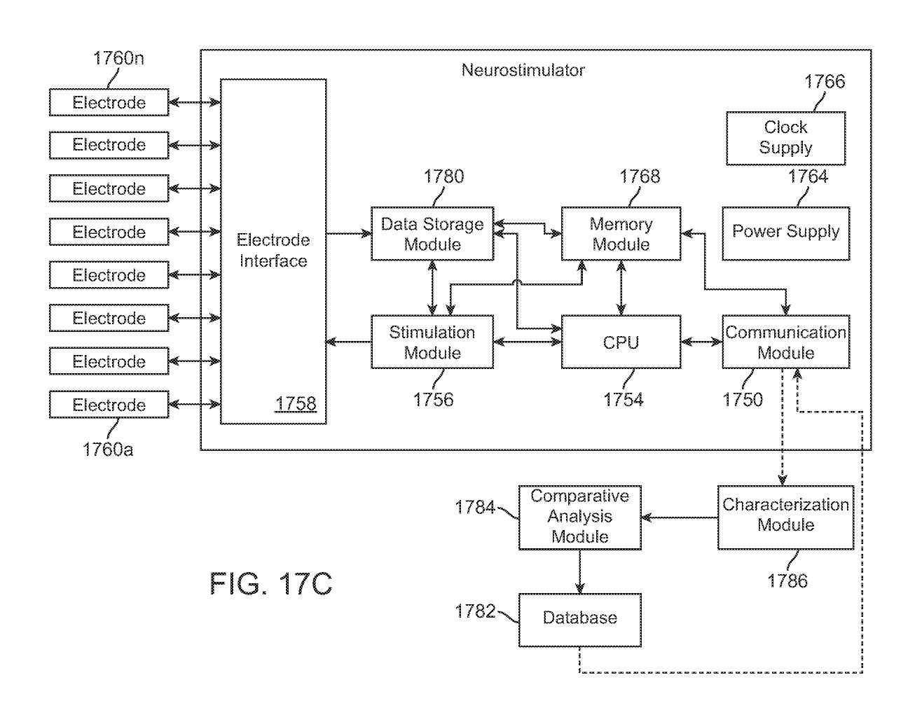

One way of configuring a system 1700 to carry out the monitoring of a target in a neural system corresponding to hippocampal pathways is described with reference to FIG. 17A.

Damage affecting the hippocampal pathways is diagnosed, for example, by a combination of neurological examinations and imaging of a patient or by a method involving EEG measurements, voltammetry or oximetry. In this example, it is assumed that the damaged cells are located either in the dentate gyrus 1440 or the CA3 region 1460 of a patient's hippocampus. (Of course, in some circumstances the interneurons may be damaged as well, which may require a different or modified systems model).

In addition to assessing damage, diagnostic tests can be used to help select a form of cell therapy to attempt to repair or reverse the damage. For example, tests can be administered to estimate the number and condition of preexisting endogenous stem cells, cytokines, growth factors, etc. at the target site. Selection of a cell therapy may also depend in part on patient specific factors, for example, if stem cells are to be implanted as part of the therapy, then more or less stem cells may be recommended based on the age of the patient (stem cells are likely to divide and differentiate more vigorously in younger patients than in older patients).

If scar tissue has developed in the region including the target by reason of the damage or otherwise, then it may be prudent to take steps to try and eliminate some of the scar tissue or prevent further scar tissue from forming or both before implementing a system for monitoring a target. Scar tissue, especially scar tissue present at the electrode-to-tissue interface, may impede the delivery of stimulation to the intended neural system and/or compromise the integrity of electrical signals sensed from the neural system. For example, pharmacological agents may be used to biochemically eradicate scar tissue or to block its further development. The agents may be delivered to the neural system locally, such as through a needle or catheter, or the electrodes may be provided with a coating containing a scar-tissue-minimizing drug.

A first electrode 1703 disposed on the distal portion of deep brain lead 1705 is positioned so that it will be in the perforant path 1720. The electrode is either configured or configurable (for example, by way of commands delivered from a neurostimulator such as the implantable neurostimulator 1707 shown) to provide electrical stimulation to the perforant path 1720 as an input for I/O characterizations of the neural system.

A second electrode 1709 disposed on the distal portion of a second deep brain lead 1711 so that it will be in the dentate gyrus 1722. The second electrode 1709 is either configured or configurable to sense electrical activity in the granule cells 1724 of the dentate gyrus 1722 when an electrical stimulation input is delivered through the stimulating electrode 1703. Again, a neurostimulator may be used to selectively configure the second electrode 1709 to record (as opposed to, for example, configuring it to deliver stimulation).

A third electrode 1713 is positioned so that it is in the CA3 region 1460 and is configured or configurable to record electrical activity corresponding to the response of the pyramidal cells 1726 to the stimulation delivered by the stimulating electrode 1703. The third electrode 1713 may be disposed on either the first or second deep brain leads 1705 or 1711 or on a third deep brain lead (not shown).

The number of leads necessary may be dictated by how precisely electrodes on a single lead can be positioned at the desired locations relative to each other. For example, if the two recording electrodes 1709 and 1713 can be spaced on a lead so that one can be positioned in the dentate gyrus 1722 and the other in the CA3 region 1728, then only one lead may be necessary for the recording electrodes. Similarly, if it is feasible to accurately position all three of the stimulating electrode 1703, the recording electrode for the dentate gyrus 1709, and the recording electrode for the CA3 region 1713, then all three electrodes may be disposed on a single deep brain lead. (In other embodiments, the electrodes may be configured to communicate with whatever components are used to provide stimulation and measure brain activity without the conductors in the deep brain leads, such as wirelessly. Thus, after the electrodes have been positioned, no leads may be necessary for communicating with the electrodes.)

Preferably, the recording electrodes 1709 and 1713 are placed as closely as possible (e.g., within the order of 130 μm) to the cell bodies of the neurons in the corresponding hippocampal regions (i.e., bodies of the granule cells 1724 of the dentate gyrus 1722 and of the pyramidal cells 1726 of the CA3 region 1728). Such placement will make it more likely that the sensed electrical activity will meaningfully correspond to the action potentials of the neurons intended to be observed and will minimize noise in the sensed signal.

Different types of electrodes may be preferred depending on whether an electrode will be configured to stimulate, record or do both. For example, if an electrode will be configured to deliver electrical stimulation, the effect of the stimulating electrode on the surrounding tissue must be considered. Chemical reactions at the electrode-to-tissue interfaces potentially can cause reactive astrocytes to develop which, in turn, can form glial scars and microglia in undesired regions. Thus, electrodes selected for stimulation should be those that minimize chemical reactions with tissue. An important property of a recording electrode will be its selectivity (interpreting which signal is being measured), so low impedance materials are desirable to keep signal-to-noise ratios favorable. Guiding the stimulating and recording electrodes 1703, 1709, and 1713 to the desired sites in the brain may be accomplished using a variety of techniques for implanting deep brain electrodes, such as stereotaxy, frameless stereotaxy, and microelectrode recording. The leads can be introduced through a burr hole 1715 and a burr hole cover (not shown) can be put in place after the electrodes have been positioned to keep the electrodes from becoming dislodged during the therapy and acquisition of baseline and therapeutic characterizations.

A block diagram illustrating some possible modules for a neurostimulator 1707 is provided in FIG. 17B. A programmable pulse generator is implemented in the neurostimulator through, inter alia, a CPU 1754, a communication module 1750, a stimulation module 1756, and an electrode interface 1758 that can be configured to be in operable communication with electrodes 1760a-1760n (although eight electrodes are shown in FIG. 17B, it will be apparent to those skilled in the art that more or less electrodes may be implicated in a particular application). A power module 1764 and clock supply module 1766 are provided for use with all possible functions of the neurostimulator, including the functions of the programmable pulse generator. The neurostimulator of FIG. 17B is also configurable to undertake I/O characterizations of neural systems and compare the results with a data storage and characterization module 1770 and a comparative analysis module 1772. A memory module 1768, which may be implemented as a separate module or as part of other modules or some combination of the two, is in communication with each of the CPU 1754, the communication module 1750, the stimulation module 1756, the data storage and characterization module 1770, and the comparative analysis module 1772. The neurostimulator also preferably has components or modules that allow bidirectional communication between the neurostimulator and external components, such as a programmer or display.

Referring again to FIG. 17A, the neurostimulator is implanted in the patient and connected to the deep brain leads 1705 and 1711 via a lead connector 1717. In this example, the neurostimulator 1707 is also configured to measure electrical activity sensed by the recording electrodes 1709 and 1713, and further includes components with which at least partially signal processing of the sensed signals can be accomplished

The neurostimulator 1707 is implanted in the patient's cranium and connected by the lead connector 1717 to the proximal ends of the deep brain leads 1705 and 1711 that extend out through the burr hole 1715. Alternatively, the neurostimulator 1707 could be positioned elsewhere in the patient's body, such as pectorally, and connected to the deep brain leads through lead extensions tunneled through the patient's neck. U.S. Pat. Nos. 6,016,449 and 6,810,285, incorporated herein in the entirety by reference, describe neurostimulators and systems including neurostimulators that can be used with the system for monitoring a target in a neural system.

Referring now to FIGS. 14, 16A-C and 18A-B, one way of undertaking a baseline characterization of the perforant path 1410-dentate gyrus 1440—CA3 region 1460 pathway is described below.

The function of the interneurons comprising the population of interneurons that is represented in the systems model as subsystem “I” 1540 in FIG. 15 is blocked so as to allow the characterization to be carried out using a simple cascade model, with subsystems “A” 1520 and “B” 1530 are isolated from subsystem “I” 1540. In this example, a drug designed to block inhibitory neurons, such as flumazenil, is delivered to the hippocampal region via a catheter. The drug delivery may be accomplished independently of any action of the neurostimulator or partially through the neurostimulator 1707 (for example, delivery of a dose of the drug may be commanded by the neurostimulator). The drug may be delivered through a catheter with an end disposed at the hippocampus to be monitored or by a reservoir near the distal end of a deep brain lead or by a drug-releasing coating on one of the electrodes 1703, 1709 and 1713.

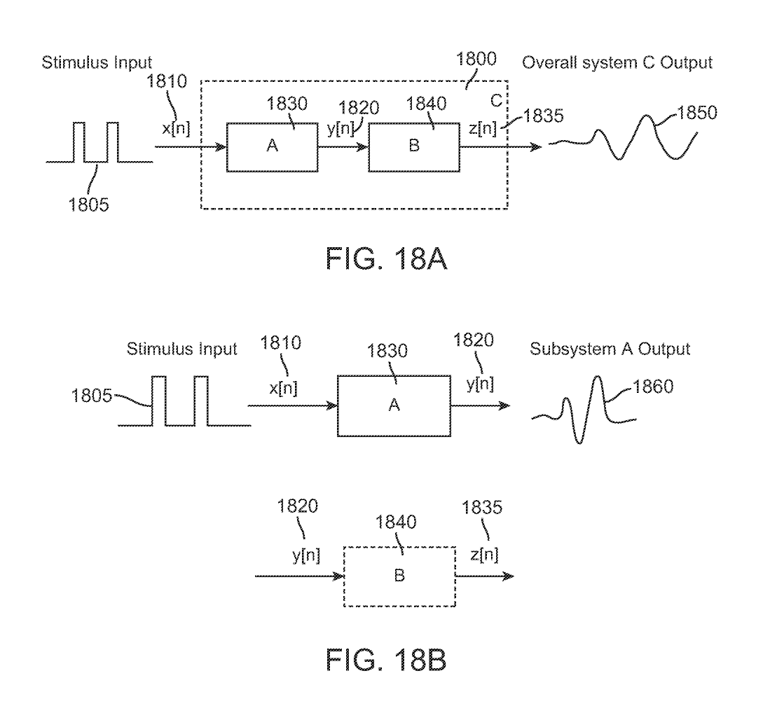

As is evident in FIG. 18A, in the simplified cascade model, the overall system input x[n] 1810 is the input to subsystem “A” 1830, and y[n] 1820 is both the output of subsystem “A” and an input to subsystem “B” 1840. The output of subsystem “B” is also the output z[n] 1835 of the overall system “C”. Subsystem “A” 1830 corresponds to the granule cells 1430 of the dentate gyrus 1440, and subsystem “B” 1840 corresponds to the pyramidal cells 1450 of the CA3 region 1460.

The neurostimulator 1707 is programmed or otherwise instructed to deliver an electrical stimulus in the form of a train of pulses at a constant frequency 1805 through the stimulating electrode 1703 to the dentate gyrus 1440 corresponding to the system input x[n] 1810. The neurostimulator 1707 may be programmed and otherwise commanded by one or more external components, such as a laptop or via an internet link to a centralized system command center or control, through the two-way communication link.

Inputs in addition to the electrical stimulus (or in lieu of it) may be used to acquire the characterizations. For example, standard clinical hippocampal-related memory tests can be administered to the patient while monitoring the output at one or more of the recording electrodes 1709 and 1713 or with a separate recording electrode that is dedicated to sensing electrophysiological responses to the tests that will be used as inputs (as opposed to recording responses to be used as outputs) in I/O characterizations. Such memory tests can include tests involving processing spatial information. Keeping track of which inputs correspond to which outputs can be accomplished manually or automatically (such as, for example, when the neurological tests are administered in a predetermined order at predetermined times).

The response y[n] 1820 of the subsystem “A” 1830 to the input(s) is sensed by the recording electrode 1709 in the dentate gyrus 1440 and is processed by the signal processing components of the neurostimulator 1707 so that it can be used in the baseline characterization (e.g., the signal from the recording electrode 1709 may be amplified, filtered to eliminate noise, monitored to account for hysteresis, etc.). The necessary signal processing may be completed exclusively with components of the neurostimulator 1707 or by a combination of neurostimulator components and external signal processing components.

The response z[n] 1835 of the overall system “C” 1800 is sensed by the recording electrode 1713 in the CA3 region 1460 and this output for the baseline characterization of the overall system “C” 1800 is processed in a manner similar to that described with respect to the output sensed at the recording electrode 1709 in the dentate gyrus 1440.

As schematically illustrated in FIGS. 18A-B, a baseline I/O characterization of the overall system “C” 1800 is calculated and a baseline I/O characterization of just subsystem “A” 1830 is calculated from the recorded output response corresponding to y[n] 1820 and the constant frequency stimulation input x[n] 1810 and based on the systems theory that has been selected. A baseline characterization for subsystem “B” 1840 can be mathematically derived from the baseline characterizations of the overall system “C” 1800 and subsystem “A” 1830 as described previously with respect to the linear system characterization (see, e.g., FIGS. 3 and 16A-C).

In this example, a linear system is assumed, although it will be apparent to those skilled in the art that a more complex system could be assumed, such as a nonlinear system using a Volterra series or neural network theory, fuzzy logic, adaptive modeling techniques, pattern matching, wavelet theory, and data mining, as well as other methods, could be employed.

The inputs and baseline output responses and baseline characterizations are stored by a storage component or storage components 1770, 1780 in the neurostimulator 1707, external components such as a database 1782 that is in communication with the neurostimulator 1707, or a combination of both. After all necessary baseline characterizations have been obtained, a predetermined cell therapy is performed. The predetermined therapy may involve implanting a quantity of stem cells at one or more locations between the stimulating electrode 1703 and the DG recording electrode 1709 and the CA3 region recording electrode 1713. (Preferably, the stem cells are implanted in a location corresponding to a pathway the therapy is intended to improve, so that, if the therapy is intended to improve the condition of the perforant path—dentate gyrus—CA3 region pathway, then the cells will be implanted between the stimulating electrode 1703 and the recording electrode 1709 in the dentate gyrus 1440. Optionally, markers may be applied to the stem cells before they are implanted to later assess stem cell differentiation (e.g., markers may be visible under a form of imaging and allow determination of whether the cells have differentiated, migrated, etc.)

Optionally, the cell therapy may further involve delivering a form of stimulation to the implant site where the stimulation is intended to encourage differentiation of stem cells into a desired type (or to encourage strengthening of desirable neural connections or to encourage inhibition of undesirable neural connections). In this example, the form of stimulation is electrical stimulation, and the neurostimulator 1707 can be configured to deliver this form of stimulation, as well as an input stimulus for the I/O characterizations. The stimulation to encourage the cell therapy may be delivered through one of the I/O characterization electrodes 1703, 1709, and 1713. However, it may be desirable to implant one or more additional electrodes in the neural system or near the neural system that are dedicated to providing stimulation for differentiation and/or recording electrical activity associated with the implant that may not necessarily be the same electrical activity that is used for the output in the I/O characterizations to monitor the target.

Electrical stimulation to encourage stem cell differentiation may include pulses or bursts of stimulation with a wide variety of variable, programmable parameters that determine such things as the type of stimulation (e.g., pulsatile, nonpulsatile (sine wave, direct current, etc.), pulse shape (biphasic or otherwise charge balanced, leading hyperpolarizing pulse, etc.), pulse amplitude, pulse frequency, pulse width, and the interstimulus interval (time between pulse trains or bursts), etc.

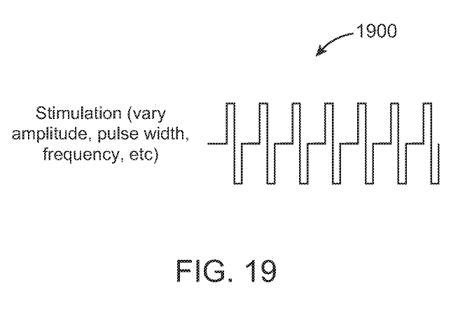

In the example shown in FIG. 19, a biphasic constant frequency stimulation pulse train 1900 is illustrated. Electrical stimulation parameters desirably are selected so as to avoid the risk of tissue damage at the electrode-to-tissue interface during delivery of stimulation to encourage stem cell differentiation. Addressing this issue may involve using charge balanced pulses delivered intermittently, rather than continuously, throughout a course of therapy. Another factor to be considered in selecting stimulation parameters is the goal of encouraging induction of the calcium ion influx that is believed to be significant in the differentiation process (electrical stimulation can cause an influx of calcium ions into the neural environment inducing stem cells to differentiate).

Monitoring of target(s) in the hippocampal neural pathways can be accomplished during and/or after the cell therapy by acquiring therapeutic characterizations using the same inputs to the overall system (and, if applicable, the inputs to any subsystems) that were used in acquiring the baseline characterizations. The details of the therapeutic characterizations (e.g., the inputs and therapeutic output responses and therapeutic characterizations are stored by a storage component or storage components 1770, 1780 in the neurostimulator 1707, external components such as a database 1782 in communication with the neurostimulator 1707, or a combination of both. If inputs other than an electrical stimulus have been used in obtaining a baseline characterization, such as clinical tests (e.g. standard memory tests, movements, auditory sound waves, light), these also should be included as inputs in acquiring the therapeutic characterizations. For instance, auditory sound waves can be used as inputs to characterize the auditory nervous system. These inputs can be stored using external components so that the same inputs can be used for multiple characterizations (e.g., baseline and therapeutic characterizations undertaken at different times).

Once a baseline characterization and at least one therapeutic characterization have been obtained, the data can be used to undertake a comparison of the baseline and therapeutic characterizations to assess whether any change is perceived. A comparison may be undertaken by displaying the baseline and therapeutic characterization results to an observer on an external display component. Alternatively, the comparison may involve further processing of the baseline and therapeutic characterization data, such as one of the cross-correlation techniques described above. A therapeutic characterization may be compared to a baseline characterization or to other therapeutic characterizations of the same PP-DG-CA3 region pathway (e.g., therapeutic characterizations taken at different times might be compared).

The neurostimulator 1707 may be configured to carry out all or a portion of the further processing; alternatively, any further processing needed to undertake the comparison may be accomplished wholly by external components (e.g., a computer with high computational capacity interacting with the database 1782) to which the neurostimulator 1707 communicates the relevant information for the comparisons. The results of the further processed comparisons may then be displayed to an observer for analysis.

Alternatively, or in addition to displaying comparison results to an observer, a system for monitoring a target in a neural system may be configured to automatically interpret a comparison and convey that interpretation to an observer. For example, a comparison may be reduced to one or more discrete values corresponding to the difference between the baseline characterization and a therapeutic characterization by the neurostimulator 1707 and/or by external components with which the neurostimulator is in communication.

The system further may be configured to compare the discrete value to a threshold (static or dynamic), and to instigate additional actions depending on whether the threshold is exceeded. The threshold may be static (e.g., based on statistical data collected from a group of similarly situated patients) or dynamic (e.g., being based on a moving average of data obtained from the patient or some other trend data). In one embodiment, the system may generate an alarm if the threshold is exceeded by a predetermined amount (e.g., a fixed amount n over the threshold), if the exceeding the threshold is likely to be an indicator that the cell therapy has had an undesirable result (e.g., stem cells differentiating into other than the desired cell type, or stem cells differentiating into an undesired cell type, such as the cell types that form teratomas).

Alternatively or additionally, the system further may be configured to compare the discrete value to a look up table where values or ranges of values correspond to different recommended actions. For example, the discrete value is “3.03” and the look up table corresponds the range of values 3.00-3.99 to a recommended action of increasing by 10% the amplitude of the stimulation applied to encourage differentiation. The system may be configured to automatically instigate the recommended action via feedback and commands to the neurostimulator 1707 or the recommendation may just be communicated to an observer who will determine whether the recommended action should be taken. Similarly, the system may be configured to notify the patient of a recommended action (or an alarm) via an external component such as a home monitoring unit (see computer (programmer or home monitoring unit) 1140 in FIG. 11). Numerous other ways of using the results of a comparison of a baseline and therapeutic characterizations to inform decisions about a cell therapy will be apparent to those skilled in the art.

Although the methods, systems and devices have been described above primarily with respect to enabling stem cell therapy, the methods, systems and devices may be used with respect to any therapy that benefits from a comparison of the measurements taken before and after stimuli are applied to encourage a change in a target population of cells or a neural circuit or portion of a neural circuit in the human body. Stimuli may include the addition or removal of stem cells to achieve the desired result in addition to stimuli comprising electrical, chemical, optical or sonic stimulation, etc. In addition, the device that accomplishes the detection function and/or stimulation function may be implanted on and/or within a patient's body in whole or in part, or may be implemented substantially with components that remain external to the patient. Additional embodiments consistent with the teachings disclosed herein are included within the scope of this disclosure.

It will be appreciated that the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A method for monitoring a target in a neural system in connection with a cell therapy for a human patient comprising:
identifying a target;
selecting a technique for acquiring input/output characterizations of the neural system;
selecting an input to use for the characterizations;
positioning a least one sensor in or near the neural system to sense an output response for the I/O characterizations;
applying the input, sensing an output and performing a baseline characterization of the neural system with a signal processor based on the applied input, the sensed output and the selected characterization technique;
introducing a change to the neural system associated with the cell therapy;
applying the input, sensing the output and performing a therapeutic characterization of the neural system with the signal processor based on the input and output and using the selected characterization technique;
displaying the results of the baseline characterization and the therapeutic characterization to permit an observer to compare the characterizations.

2. The method of claim 1 wherein the target includes a population of a particular neuronal cell type within the neural system.

3. The method claim 2 wherein the neuronal cell type is one of the group including excitatory neurons and inhibitory neurons.

4. The method of claim 2 wherein the neuronal cell type is one of the group including granule cells, pyramidal cells, and interneurons.

5. The method claim 1 wherein the selected technique for acquiring the characterizations is based one of linear or nonlinear systems theory.

6. The method of claim 5 wherein the selected technique for acquiring the characterizations is further based on one or more systems models.

7. The method of claim 6 wherein the systems models are selected from the group including a feedback model, an addition model, and a cascade model.

8. The method of claim 1 wherein positioning at least one sensor includes positioning a first sensor at a first location in a hippocampal pathway in the patient's brain and positioning a second sensor at a second location in the patient's brain.

9. The method of claim 8 wherein each of the first and second sensors is an electrode configured to sense electrical activity from the patient's brain.

10. The method of claim 1 wherein each of the first and second sensors is an electrode configured to sense electrical activity from the patient's brain and further comprising positioning a stimulating electrode in or near the neural system to deliver the input for the I/O characterizations, generating an electrical stimulus corresponding to the selected input, and delivering the generated electrical stimulus through the stimulating electrode.

11. The method of claim 10 wherein the selected technique for acquiring the I/O characterizations is based one of linear or nonlinear systems theory and is further based on one or more systems models.

12. The method of claim 11 wherein the one or more systems models are from the group including a feedback model, an addition model, and a cascade model.

13. The method of claim 1 wherein introducing a change comprises at least implanting a quantity of stem cells into a region within the neural system or associated with the neural system.

14. The method of claim 13 wherein introducing a change further comprises delivering a form of stimulation to the implanted quantity of stem cells.

15. The method of claim 14 wherein the form of stimulation is electrical stimulation.

16. The method of claim 1 wherein introducing a change comprises at least delivering a burst of electrical stimulation to a quantity of stem cells.

17. The method of claim 16 wherein delivering a burst of electrical stimulation further comprises delivering a burst of electrical stimulation to a region of the brain including stem cells with a stimulation electrode in communication with a pulse generator that generates the burst of electrical stimulation.

18. A system for monitoring a target in a neural system of a human patient before and after a change is introduced to the system, the system comprising:
an implantable sensor adapted to sense electrical activity from a first location in the neural system;
an implantable device configured to be in operable communication with the implantable sensor and adapted to:
measure a response of the sensor to a predetermined input,
process the response together with the predetermined input to calculate a plurality of input/output characterizations of the neural system based on each measured output response and the predetermined input; and
communicate the plurality of input/output characterizations to at least one component external of the patient;
a comparator adapted to generate at least one comparison result based on comparing a baseline input/output characterization of the neural system to at least one therapeutic input/output characterization acquired during or after a change is introduced to the neural system; and
a display configured to display to the at least one comparison result.

19. The system of claim 18, further comprising a deep brain lead having a lead body, a distal portion, a proximal portion, and at least one insulated conductor extending through the lead body; the lead adapted to carry the sensor at the distal portion and to attach to the implantable device at the proximal portion and to connect the sensor through at least one insulated conductor so that the sensor is in operable communication with the implantable device.

20. The system of claim 18, further comprising an implantable stimulating electrode adapted to deliver a form of electrical stimulation from a second location in the neural system wherein the delivered electrical stimulation comprises at least part of the predetermined input.

21. The system of claim 20 herein the implantable device further is adapted to generate the form of electrical stimulation and to introduce the electrical stimulation to the stimulating electrode.

22. The system of claim 21 further comprising a deep brain lead having a lead body, distal portion, a proximal portion, and at least one insulated conductor extending through the lead body; the lead adapted to carry the stimulating electrode at the distal portion and to attach to the implantable device at the proximal portion and to connect the stimulating electrode through at least one insulated conductor so that the stimulating electrode is in operable communication with the implantable device.
